(19) United States
(12) Patent Application Publication
(10) Pub. No.: US 2024/0002491 A1
(43) Pub. Date: Jan. 4, 2024

(54) METHODS FOR SELECTING PATIENTS FOR TREATMENT WITH AN NGF ANTAGONIST

(71) Applicant: Regeneron Pharmaceuticals, Inc., Tarrytown, NY (US)

(72) Inventor: Stephen DIMARTINO, Scarsdale, NY (US)

(21) Appl. No.: 18/307,309

(22) Filed: Apr. 26, 2023

Related U.S. Application Data
(60) Provisional application No. 63/335,409, filed on Apr. 27, 2022.

(51) Int. Cl. C07K 16/22; G01N 33/573; A61P 29/00; A61P 19/02

(52) U.S. Cl. CPC C07K 16/22; G01N 33/573; A61P 29/00; A61P 19/02; G01N 2333/916; G01N 2800/52; C07K 2317/76; A61K 2039/505

(57) ABSTRACT

Methods and compositions for treating pain associated with osteoarthritis of the knee and/or hip are disclosed. In certain aspects, the subject to be treated is selected on the basis of the number of joints that exhibit osteoarthritis and/or on the basis of levels of alkaline phosphatase in the subject. In certain aspects, the subject is treated with an anti-NGF antibody such as fasinumab.

### CROSS-REFERENCE TO RELATED APPLICATIONS

This application claims priority to U.S. Provisional Patent Application No. 63/335,409, filed Apr. 27, 2022, the contents of which are incorporated by reference herein.

### REFERENCE TO SEQUENCE LISTING SUBMITTED ELECTRONICALLY

The content of the electronically submitted sequence listing in XML format (Name: 741141_RGN9-004_ST26.xml; Size: 24,593 bytes; Created: Aug. 31, 2023) is incorporated herein by reference in its entirety.

### FIELD OF THE INVENTION

Disclosed herein are methods and compositions related to the treatment, reduction, or improvement of pain in subjects with osteoarthritis. Also disclosed herein are methods for mitigating risk of arthropathies and joint replacement in subjects with osteoarthritis, and methods of selecting subjects for treatment with a nerve growth factor (NGF) antagonist, such as an anti-NGF antibody.

### BACKGROUND

Chronic musculoskeletal pain affects a large portion of the global population. A significant cause of chronic musculoskeletal pain is due to osteoarthritis (OA). Osteoarthritis is a progressive, chronic disease which is caused by the breakdown and loss of cartilage of the joints which leads to pain in the hips, knees, hands, feet, and spine. It is characterized by focal areas of loss of articular cartilage in synovial joints accompanied by subchondral bone changes, osteophyte formation at the joint margins, thickening of the joint capsule and mild synovitis. Symptoms and disability increase with increasing age. The prevalence of OA in patients aged 65 and older is 60% in men and 70% in women, and continually rising.

Many patients with acute and chronic pain due to OA do not receive adequate pain relief despite the wide variety of analgesic medications that are currently available. Analgesics such as non-steroidal anti-inflammatory drugs (NSAIDs) provide only modest benefits, as there are a significant number of patients who are intolerant to or do not get adequate pain relief from the currently available treatments. Other analgesics such as opioids are typically associated with the unacceptable risks of toxicity or dependence. Inadequate pain relief has a profound impact on the quality of life for millions of people worldwide with an associated substantial cost to society, including healthcare cost and loss of productivity.

Neurotrophins are a family of peptide growth factors that play a role in the development, differentiation, survival and death of neuronal and non-neuronal cells. One such neurotrophin is nerve growth factor (NGF). The NGF/tyrosine kinase type 1 (TrkA) receptor system appears to play a major role in the control of pain. Administration of NGF has been shown to provoke pain in both rodents (Lewin et al., (1994), Eur. J. Neurosci 6:1903-1912) and humans (McArthur et al., (2000), Neurology 54:1080-1088), while NGF antagonists have been shown to prevent hyperalgesia and allodynia in animal models of neuropathic and chronic inflammatory pain (Ramer et al., (1999) Eur J Neurosci 11:837-846). Humans with mutations in TrkA (hereditary sensory and autonomic neuropathy IV) or NGF (hereditary sensory and autonomic neuropathy V) have been identified with a loss of deep pain perception (Indo et al., (1996), Nature Genetics, 13:485-488); Einarsdottir et al., (2004), Human Molecular Genetics 13:799-805). In addition, NGF is known to be elevated in the synovial fluid of patients with rheumatoid arthritis and other types of arthritis (Aloe, L. et. al., (1992), Arthritis Rheum 35:351-355; Halliday, D. A., (1998), Neurochem Res. 23:919-922), and to be up regulated in injured and inflamed tissues in conditions such as cystitis, prostatitis, and chronic headache (Lowe et. al., (1997), Br. J. Urol. 79:572-577; Miller, L. J., et. al., (2002), Urology 59:603-608; Sarchielli et al., (2001), Neurology 57:132-134).

Biologic agents that specifically block NGF to treat pain may obviate many of the side effects of currently used analgesic medications such as opioids and NSAIDs. Anti-NGF antibodies have been shown to produce significant pain relief and functional improvement in patients with osteoarthritis of the knee and/or hip; however, data from clinical studies of anti-NGF antibodies suggested an increased risk of joint destruction such as destructive arthropathy (Bannwarth et al., (2014), Drugs 74:619-626).

Accordingly, there remains an unmet medical need for alternative pain relief treatment options for subjects with osteoarthritis or knee and/or hip that mitigate the risk of joint destruction or joint replacement in subjects while providing adequate pain relief.

### SUMMARY OF THE DISCLOSURE

In one aspect, the present disclosure provides methods for treating or reducing pain associated with osteoarthritis of the knee and/or hip in a subject with an NGF antagonist, and methods of mitigating the risk of joint destruction in a subject being treated with an NGF antagonist for pain associated with osteoarthritis of the knee and/or hip. In some embodiments, the method comprises selecting a subject on the basis of the number of large joints at baseline that exhibit osteoarthritis. In some embodiments, the method comprises selecting a subject on the basis of the level of change in alkaline phosphatase level from baseline to a timepoint after the start of treatment. In some embodiments, patients are selected on the basis of a combination thereof.

In one aspect, the methods of the present disclosure comprise:
(a) selecting a subject having osteoarthritis of the knee or hip, wherein the subject to be treated has no more than 2 large joints that exhibit osteoarthritis, wherein the large joints are selected from the group consisting of knee joint, hip joint, and shoulder joint;
(b) administering to the subject one or more doses of an NGF antagonist;
(c) determining whether the subject is a candidate for continued treatment with the NGF antagonist, comprising comparing the level of alkaline phosphatase in the subject at a timepoint after the start of treatment with the NGF antagonist to a baseline level of alkaline phosphatase in the subject prior to or at the start of treatment, wherein a subject is identified as a candidate for continued treatment if the subject does not have an increase in the level of alkaline phosphatase that is above a threshold value; and
(d) administering to the subject who is identified as a candidate for continued treatment one or more additional doses of the NGF antagonist.

In some embodiments, the threshold value in step (c) is a 15-point (e.g., 15 U/L) increase in the level of alkaline phosphatase, relative to the baseline level of alkaline phosphatase. In some embodiments, the threshold value in step (c) is a 10-point increase in the level of alkaline phosphatase, relative to the baseline level of alkaline phosphatase. In some embodiments, the threshold value in step (c) is a 5-point increase in the level of alkaline phosphatase, relative to the baseline level of alkaline phosphatase. In some embodiments, the baseline level of alkaline phosphatase is an average of two or more measurements of alkaline phosphatase for the subject, wherein each measurement is taken prior to or at the start of treatment.

In some embodiments, in step (c) the timepoint is about 8 weeks to about 16 weeks from the start of treatment with the NGF antagonist.

In some embodiments, step (c) further comprises measuring the level of pain in the subject and identifying the subject as a candidate for continued treatment if the subject exhibits a decrease in pain at a timepoint after the start of treatment relative to a baseline level of pain in the subject prior to or at the start of treatment. In some embodiments, the level of pain is measured using:
(a) the Western Ontario and McMaster Universities Osteoarthritis Index (WOMAC) pain subscale score;
(b) the Numeric Rating Scale (NRS) for knee and/or hip pain intensity; and/or
(c) the Patient Global Assessment (PGA) of knee and/or hip pain.

In some embodiments, the subject is identified as a candidate for continued treatment if the subject exhibits a ≥30% improvement in WOMAC pain subscale score relative to the baseline level.

In some embodiments, in step (a) the number of joints that exhibit osteoarthritis is determined by X-ray.

In some embodiments, the method further comprises:
(e) comparing the level of alkaline phosphatase in the subject at a second timepoint to the baseline level of alkaline phosphatase in the subject, wherein the second timepoint is after the administration of the one or more additional doses of the NGF antagonist according to step (d).

In some embodiments, the second timepoint is at least 8 weeks after the administration of the one or more additional doses of the NGF antagonist.

In another aspect, the methods of the present disclosure comprise:
(a) administering to the subject an initial dose of an NGF antagonist;
(b) administering to the subject one or more secondary doses of the NGF antagonist, wherein each secondary dose is administered 4 weeks or 8 weeks after the immediately preceding dose;
(c) obtaining a measurement of alkaline phosphatase level in the subject at a timepoint from 8 weeks to 16 weeks after the administration of the initial dose; and
(d) administering to the subject one or more tertiary doses of the NGF antagonist only if the subject's alkaline phosphatase level in step (c) is not increased above a threshold value, relative to a baseline level of alkaline phosphatase in the subject prior to or at the start of treatment; wherein each tertiary dose is administered 4 weeks or 8 weeks after the immediately preceding dose.

In some embodiments, the threshold value in step (c) is a 20-point (e.g., 20 U/L) increase in the level of alkaline phosphatase, relative to the baseline level of alkaline phosphatase. In some embodiments, the threshold value in step (c) is a 15-point (e.g., 15 U/L) increase in the level of alkaline phosphatase, relative to the baseline level of alkaline phosphatase. In some embodiments, the threshold value in step (c) is a 10-point (e.g., 10 U/L) increase in the level of alkaline phosphatase, relative to the baseline level of alkaline phosphatase. In some embodiments, the threshold value in step (c) is a 5-point (e.g., 5 U/L) increase in the level of alkaline phosphatase, relative to the baseline level of alkaline phosphatase. In some embodiments, the baseline level of alkaline phosphatase is an average of two or more measurements of alkaline phosphatase for the subject, wherein each measurement is taken prior to or at the start of treatment.

In some embodiments, in step (c) the timepoint is about 8 weeks to about 16 weeks from the start of treatment with the NGF antagonist.

In some embodiments, the alkaline phosphatase is serum alkaline phosphatase. In some embodiments, the alkaline phosphatase is bone-specific alkaline phosphatase. In some embodiments, the alkaline phosphatase is measured using an enzymatic assay.

In another aspect, the methods of the present disclosure comprise:
(a) selecting a subject having osteoarthritis of the knee or hip, wherein the subject to be treated has no more than 2 large joints that exhibit osteoarthritis, wherein the large joints are selected from the group consisting of knee joint, hip joint, and shoulder joint; and
(b) administering to the subject an NGF antagonist, wherein the NGF antagonist is administered every four weeks (Q4W) or every eight weeks (Q8W).

In some embodiments, in step (a) the number of joints that exhibit osteoarthritis is determined by X-ray.

In some embodiments, a subject to be treated according to the methods disclosed herein does not have a pre-existing subchondral insufficiency fracture (SIF) or osteonecrosis.

In some embodiments, a subject to be treated according to the methods disclosed herein the subject is resistant, non-responsive, or inadequately responsive to treatment with a standard analgesic, or wherein the subject has an intolerance to standard analgesic therapy. In some embodiments, the standard analgesic therapy is acetaminophen/paracetamol, a nonsteroidal anti-inflammatory drug (NSAID), an opioid, or a combination thereof.

In some embodiments, a subject to be treated according to the methods disclosed herein has an osteoarthritis polygenic risk score (OA-PRS) that is less than a threshold OA-PRS, wherein the OA-PRS comprises a weighted aggregate of a plurality of genetic variants associated with osteoarthritis.

In some embodiments, the NGF antagonist is an anti-NGF antibody or antigen-binding fragment thereof. In some embodiments, the anti-NGF antibody or antigen-binding fragment thereof comprises three heavy chain complementarity determining region (HCDR) sequences (HCDR1, HCDR2, and HCDR3) comprising the amino acid sequences of SEQ ID NOs: 4, 6, and 8, respectively, and three light chain complementarity determining (LCDR) sequences (LCDR1, LCDR2, and LCDR3) comprising the amino acid sequences of SEQ ID NOs: 12, 14, and 16, respectively. In some embodiments, the anti-NGF antibody or antigen-binding fragment thereof comprises a heavy chain variable region (HCVR) comprising the amino acid sequence of SEQ ID NO:2 and a light chain variable region (LCVR) comprising the amino acid sequence of SEQ ID NO:10. In some embodiments, the anti-NGF antibody or antigen-binding fragment thereof comprises a heavy chain comprising the amino acid sequence of SEQ ID NO:17 and/or a light chain comprising the amino acid sequence of SEQ ID NO:18. In some embodiments, the anti-NGF antibody is fasinumab.

In some embodiments, the anti-NGF antibody or antigen-binding fragment thereof is tanezumab or fulranumab.

In some embodiments, the anti-NGF antibody or antigen-binding fragment thereof is administered every four weeks (Q4W) or every eight weeks (Q8W).

In some embodiments, the anti-NGF antibody or antigen-binding fragment thereof is administered at a dose from 0.5 mg to 10 mg. In some embodiments, the anti-NGF antibody or antigen-binding fragment thereof is administered at a dose of about 1 mg.

The present disclosure also provides pharmaceutical compositions for use in any of the methods or embodiments of the present disclosure. In some embodiments, the pharmaceutical composition comprises an anti-NGF antibody or antigen-binding fragment thereof, e.g., an anti-NGF antibody comprising three heavy chain complementarity determining region (HCDR) sequences (HCDR1, HCDR2, HCDR3) comprising SEQ ID NOs: 4, 6 and 8, respectively, and three light chain complementarity determining (LCDR) sequences (LCDR1, LCDR2, LCDR3) comprising SEQ ID NOs: 12, 14 and 16, respectively.

The present disclosure also provides for the use of an NGF antagonist for the preparation of a medicament for the treatment of pain associated with OA of the knee and/or hip as disclosed herein. In some embodiments, the NGF antagonist is an anti-NGF antibody or antigen-binding fragment thereof, e.g., an anti-NGF antibody comprising three heavy chain complementarity determining region (HCDR) sequences (HCDR1, HCDR2, HCDR3) comprising SEQ ID NOs: 4, 6 and 8, respectively, and three light chain complementarity determining (LCDR) sequences (LCDR1, LCDR2, LCDR3) comprising SEQ ID NOs: 12, 14 and 16, respectively.

### BRIEF DESCRIPTION OF THE FIGURES

FIG. 1: ISS subgroup analysis for the percentage of patients who had joint replacements (JR) versus the number of large joints with OA at baseline (ISS total population, 1-2 joints subgroup, 3-4 joints subgroup, and 5-6 joints subgroup) by treatment arm (placebo, fasinumab at 1 mg Q8W, and fasinumab at 1 mg Q4W).

FIG. 2: ISS subgroup analysis for the percentage of patients who had an adjudicated arthropathy (AA) versus the number of large joints with OA at baseline (ISS total population, 1-2 joints subgroup, 3-4 joints subgroup, and 5-6 joints subgroup) by treatment arm (placebo, fasinumab at 1 mg Q8W, and fasinumab at 1 mg Q4W).

FIG. 3: ISS subgroup analysis for the percentage of patients who had a destructive arthropathy (DA) versus the number of large joints with OA at baseline (ISS total population, 1-2 joints subgroup, 3-4 joints subgroup, and 5-6 joints subgroup) by treatment arm (placebo, fasinumab at 1 mg Q8W, and fasinumab at 1 mg Q4W).

FIG. 4: Forest plot showing change from baseline in WOMAC pain subscale score at 16 weeks for the following fasinumab treatment groups versus placebo: 1 mg Q4W and 1-2 large joints with OA at baseline; 1 mg Q4W and 3-4 large joints with OA at baseline; 1 mg Q4W and 5-6 large joints with OA at baseline; 1 mg Q8W and 1-2 large joints with OA at baseline; 1 mg Q8W and 3-4 large joints with OA at baseline; and 1 mg Q8W and 5-6 large joints with OA at baseline.

FIG. 5: ISS laboratory results for mean change (SE) from baseline in alkaline phosphatase (U/L) in patients without AA, with DA, or with non-DA AA, for each the treatment cohorts placebo, NSAID, fasinumab 1 mg Q4W, fasinumab 1 mg Q8W, and fasinumab high dose (which includes 3 mg Q4W, 6 mg Q4W, 9 mg Q8W, and 9 mg Q4W).

FIG. 6: ISS rates of DA for patients at 16 weeks above and below various cutoffs for change in alkaline phosphatase (≥10, ≥15, or ≥20 point (U/L) increase) for patients treated with placebo, fasinumab at 1 mg Q8W, or fasinumab at 1 mg Q4W.

FIG. 7: ISS rates of AA for patients at 16 weeks above and below various cutoffs for change in alkaline phosphatase (≥10, ≥15, or ≥20 point (U/L) increase) for patients treated with placebo, fasinumab at 1 mg Q8W, or fasinumab at 1 mg Q4W.

FIGS. 8A-8F: Impact of combining risk mitigation approaches on risk reduction. FIG. 8A-8B: The percentage of patients with JR, for total ISS population and for OA joint burden subgroups (1-2 joints, 1-3 joints, and 1-4 joints), following exclusion of patients with change in alkaline phosphatase at or above the designated cutoff values at any time between weeks 8 and 16 of treatment with fasinumab 1 mg Q8W (A) or 1 mg Q4W (B). FIG. 8C-8D: The percentage of patients with AA, for total ISS population and for OA joint burden subgroups (1-2 joints, 1-3 joints, and 1-4 joints), following exclusion of patients with change in alkaline phosphatase at or above the designated cutoff values at any time between weeks 8 and 16 of treatment with fasinumab 1 mg Q8W (C) or 1 mg Q4W (D). FIG. 8E-8F: The percentage of patients with DA, for total ISS population and for OA joint burden subgroups (1-2 joints, 1-3 joints, and 1-4 joints), following exclusion of patients with change in alkaline phosphatase at or above the designated cutoff values at any time between weeks 8 and 16 of treatment with fasinumab 1 mg Q8W (E) or 1 mg Q4W (F).

### DETAILED DESCRIPTION

Before the invention disclosed herein is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

### Definitions

Unless defined otherwise, all technical and scientific terms used herein have the same meaning as commonly understood by one of ordinary skill in the art to which this invention belongs.

All patents, applications and non-patent publications mentioned herein are incorporated herein by reference in their entireties.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

The terms “NGF,” “hNGF,” and the like, as used herein, are intended to refer to nerve growth factor, and in particular, to human nerve growth factor, the amino acid sequence of which is shown as SEQ ID NO:20 and which is encoded by the nucleic acid sequence shown as SEQ ID NO: 19. Unless specifically designated as being from a non-human species, the term “NGF”, as used herein, shall be understood to mean human NGF.

As used herein, an “NGF antagonist” refers to any agent that binds to or interacts with NGF and inhibits the normal biological function of NGF in vitro or in vivo. Non-limiting examples of categories of NGF antagonists include small molecule NGF antagonists, anti-NGF aptamers, peptide-based NGF antagonists (e.g., “peptibody” molecules), and antibodies or antigen-binding fragments of antibodies that specifically bind human NGF. In a specific embodiment, the NGF antagonist is fasinumab.

The term “antibody,” as used herein, refers to immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL1). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the disclosure, the FRs of the anti-NGF antibody (or antigen-binding portion thereof) may be identical to the human germline sequences, or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present disclosure include: (i) VH-CH1; (ii) VH-CH2; (iii) VH-CH3; (iv) VH-CH1-CH2; (v) VH-CH1-CH2-CH3; (vi) VH-CH2-CH3; (vii) VH-CL; (viii) VL-CH1; (ix) VL-CH2; (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present disclosure may comprise a homo-dimer or hetero-dimer (or other multimer) of any of the variable and constant domain configurations listed above in non-covalent association with one another and/or with one or more monomeric VH or VL domain (e.g., by disulfide bond(s)).

The term “antibody,” as used herein, also includes multispecific (e.g., bispecific) antibodies. A multispecific antigen-binding fragment of an antibody will typically comprise at least two different variable domains, wherein each variable domain is capable of specifically binding to a separate antigen or to a different epitope on the same antigen. Any multispecific antibody format, may be adapted for use in the context of an antigen-binding fragment of an antibody of the present disclosure using routine techniques available in the art. For example, the present disclosure includes methods comprising the use of bispecific antibodies wherein one arm of an immunoglobulin is specific for NGF or a fragment thereof, and the other arm of the immunoglobulin is specific for a second therapeutic target or is conjugated to a therapeutic moiety. Exemplary bispecific formats that can be used in the context of the present disclosure include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED) body, leucine zipper, Duobody, IgG1/IgG2, dual acting Fab (DAF)-IgG, and Mab2 bispecific formats (see, e.g., Klein et al., 2012, mAbs 4:6, 1-11, and references cited therein, for a review of the foregoing formats). Bispecific antibodies can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

In some embodiments, the antibodies used in the methods of the present disclosure are human antibodies. The term “human antibody,” as used herein, is intended to include antibodies having variable and constant regions derived from human germline immunoglobulin sequences. The human antibodies of the disclosure may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present disclosure may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al., (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

An “isolated antibody,” as used herein, refers to an antibody that has been identified and separated and/or recovered from at least one component of its natural environment. For example, an antibody that has been separated or removed from at least one component of an organism, or from a tissue or cell in which the antibody naturally exists or is naturally produced, is an “isolated antibody.” An isolated antibody also includes an antibody in situ within a recombinant cell. Isolated antibodies are antibodies that have been subjected to at least one purification or isolation step. According to certain embodiments, an isolated antibody may be substantially free of other cellular material and/or chemicals.

The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” NGF, as used in the context of the present disclosure, includes antibodies that bind NGF or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM, less than about 0.5 nM, less than 0.1 nM, less than 1.0 pM, or less than 0.5 pM, as measured in a surface plasmon resonance assay. An isolated antibody that specifically binds human NGF may, however, have cross-reactivity to other antigens, such as NGF molecules from other (non-human) species.

The term “surface plasmon resonance,” as used herein, refers to an optical phenomenon that allows for the analysis of real-time interactions by detection of alterations in protein concentrations within a biosensor matrix, for example using the BIAcore™ system (Biacore Life Sciences division of GE Healthcare, Piscataway, NJ).

The term “K_{D},” as used herein, refers to the equilibrium dissociation constant of a particular antibody-antigen interaction.

The term “epitope” refers to an antigenic determinant that interacts with a specific antigen binding site in the variable region of an antibody molecule known as a paratope. A single antigen may have more than one epitope. Thus, different antibodies may bind to different areas on an antigen and may have different biological effects. Epitopes may be either conformational or linear. A conformational epitope is produced by spatially juxtaposed amino acids from different segments of the linear polypeptide chain. A linear epitope is one produced by adjacent amino acid residues in a polypeptide chain. In certain circumstance, an epitope may include moieties of saccharides, phosphoryl groups, or sulfonyl groups on the antigen.

As used herein, the terms “treat,” “treating,” or the like, mean to alleviate symptoms, eliminate the causation of symptoms either on a temporary or permanent basis, or to prevent or slow the appearance of symptoms of the named disorder or condition.

In some embodiments, “a subject in need thereof” refers to a human or non-human mammal that exhibits one or more symptoms of osteoarthritis pain (e.g., chronic OA of the knee and/or hip), and/or who has been diagnosed with osteoarthritis or pain associated with osteoarthritis (e.g., chronic OA of the knee and/or hip). In certain embodiments, the methods disclosed herein may be used to treat patients that have OA of the knee and/or hip with Kellgren-Lawrence [K-L] grading of on a scale of 0-4 and/or moderate-to-severe pain in one or more joints, defined as a WOMAC pain subscale score of ≥4. In certain embodiments, “a subject in need thereof” refers to a patient suffering from knee and/or hip pain, who has a history of inadequate pain relief from standard analgesic therapy (e.g., no significant pain reduction after administration of the standard analgesic therapy for an average of 4 days/week during a 4 week period), or intolerance to standard analgesic therapy. As used herein, the term “inadequate pain relief” refers to an unacceptable level of pain relief experienced by subjects after pain relief treatment, such as treatment with a standard analgesic. For example, subjects with inadequate pain relief may find that they cannot go about conducting normal daily activities due to the pain level index. The term “inadequate pain relief” also refers to an unacceptable reduction in pain and/or unacceptable improvement in pain after pain relief treatment, such as treatment with a standard analgesic. The term “intolerance to standard analgesic therapy,” as used herein, refers to subjects or patients who exhibit an adverse event or side effect after treatment with the standard analgesic, such as for example an allergic reaction to a standard analgesic. The term “resistant, non-responsive, or inadequately responsive to a standard analgesic”, as used herein, refers to subjects or patients with knee and/or hip pain who have been treated with a standard analgesic (for example, an NSAID), and wherein the standard analgesic does not have a sufficient therapeutic effect.

In some embodiments, “a subject in need thereof” refers to a subject who, prior to treatment, exhibits (or has exhibited) one or more pain-associated parameters, such as but not limited to: (a) Western Ontario and McMaster Universities Osteoarthritis Index (WOMAC) pain score; (b) WOMAC physical function subscale score; (c) Patient Global Assessment (PGA) score; (d) the knee and/or hip pain Numerical Rating Scale (NRS) score; (e) the short form health survey (SF-36) subscale score; (f) the EuroQoL 5 Dimensions 5 Level Questionnaire (EQ-5D-5L); or (g) use of rescue medication for knee and/or hip pain.

As used herein, the term “treatment-emergent adverse event” (TEAE) refers to an adverse event that was not present at baseline or that represents the exacerbation of a pre-existing condition which occurs while being treated (e.g., with an NGF antagonist). In some embodiments, the adverse event is an adjudicated arthropathy. The term “adjudicated arthropathy” as defined herein is an umbrella term that encompasses the following conditions: rapidly progressive OA type 1 or 2, subchondral insufficiency fractures, and primary osteonecrosis. In some embodiments, rapidly progressive OA type 1 (RPOA-1) is defined as joint space narrowing measured on X-ray exceeding pre-specified thresholds and accompanied by articular cartilage loss observed on MRI. In some embodiments, rapidly progressive OA type 2 (RPOA-2) is defined as changes in bone structure principally observable on MRI, although may be observed on X-rays.

In some embodiments, an adjudicated arthropathy is a destructive arthropathy. As used herein, “destructive arthropathy” refers to abnormal bone fragmentation, destruction, or fracture over a short period of time, including near-total collapse of an articular surface, and often associated with subluxation or malalignment, which are observed by X-ray radiography and which are features inconsistent with radiographic findings typically observed in conventional advanced OA.

### Methods of Mitigating the Risk of Joint Destruction

In one aspect, disclosed herein are methods for mitigating the risk of joint destruction (e.g., destructive arthropathy) or joint replacement in a subject having OA who is being treated with (or will be treated with) an NGF antagonist, e.g., an anti-NGF antibody or antigen binding fragment thereof as disclosed herein. Further disclosed herein are methods for identifying or selecting a subject having OA who is a suitable candidate for treatment with an NGF antagonist, e.g., an anti-NGF antibody or antigen binding fragment thereof as disclosed herein.

Selection on the Basis of Joints that Exhibit Osteoarthritis and/or Joint Arthropathy

In some embodiments, the method comprises selecting a subject having osteoarthritis of the knee or hip on the basis of the number of large joints that exhibit osteoarthritis (e.g., prior to the start of treatment with an anti-NGF antibody). In some embodiments, the subject to be treated has 1-3 large joints that exhibit osteoarthritis. In some embodiments, the subject to be treated has 1-2 large joints that exhibit osteoarthritis. In some embodiments, the subject to be treated has no more than 3 large joints that exhibit osteoarthritis. In some embodiments, the subject to be treated has no more than 2 large joints that exhibit osteoarthritis. In some embodiments, the large joints are selected from the group consisting of knee joint, hip joint, and shoulder joint. In some embodiments, the number of joints that exhibit osteoarthritis is determined by X-ray. In some embodiments, the number of joints that exhibit osteoarthritis is determined by magnetic resonance imaging (MRI).

In some embodiments, the method comprises selecting a subject on the basis of the number of large joints (knee, hip, and/or shoulder joints) that have an OA grade of mild, moderate, or severe. In some embodiments, the subject to be treated has 1-4 large joints that exhibit mild, moderate, or severe osteoarthritis. In some embodiments, the subject to be treated has 1-3 large joints that exhibit mild, moderate, or severe osteoarthritis. In some embodiments, the subject to be treated has 1-2 large joints that exhibit mild, moderate, or severe osteoarthritis. In some embodiments, the subject to be treated has no more than 4 large joints that exhibit mild, moderate, or severe osteoarthritis. In some embodiments, the subject to be treated has no more than 3 large joints that exhibit mild, moderate, or severe osteoarthritis. In some embodiments, the subject to be treated has no more than 2 large joints that exhibit mild, moderate, or severe osteoarthritis.

In some embodiments, the method comprises selecting a subject on the basis of the number of large joints (knee, hip, and/or shoulder joints) that have a K-L grade of ≥2. The Kellgren-Lawrence [K-L] grading system uses plain radiographs and provides grades as follows: Grade 0, No radiographic features of osteoarthritis; Grade 1, Possible joint space narrowing (normal joint space is at least 2 mm at the superior acetabulum) and osteophyte formation; Grade 2, Definite osteophyte formation with possible joint space narrowing; Grade 3, Multiple osteophytes, definite joint space narrowing, sclerosis and possible bony deformity; Grade 4, Large osteophytes, marked joint space narrowing, severe sclerosis, and definite bony deformity. In some embodiments, the subject to be treated has 1-3 large joints that have a K-L grade of ≥2. In some embodiments, the subject to be treated has 1-2 large joints that have a K-L grade of ≥2. In some embodiments, the subject to be treated has no more than 3 large joints that have a K-L grade of ≥2. In some embodiments, the subject to be treated has no more than 2 large joints that have a K-L grade of ≥2.

In some embodiments, the method comprises selecting a subject who does not have a pre-existing joint arthropathy, or history of joint arthropathy, that would place the subject at increased risk of joint destruction. In some embodiments, the method comprises selecting a subject who does not have a history of rapidly progressive OA (RPOA), subchondral insufficiency fracture (SIF), or osteonecrosis (ON). In some embodiments, the subject does not have a pre-existing subchondral insufficiency fracture (SIF) or osteonecrosis.

### Selection on the Basis of Bone Formation Markers

In some embodiments, the method comprises selecting the subject on the basis of one or more bone formation markers, such as a baseline level of a bone formation marker that is above a threshold level, or a change in the subject's level of the bone formation marker from baseline to a timepoint after the start of treatment with an NGF antagonist.

In some embodiments, a bone formation marker is an enzyme, protein, or protein derivative that is associated with or a product of osteoblast function. Bone formation markers include, but are not limited to, alkaline phosphatase (total alkaline phosphatase or bone-specific alkaline phosphatase), osteocalcin, and propeptides of type I procollagen (procollagen type I N-terminal propeptide (PINP) and procollagen type I C-terminal propeptide (PICP)). In some embodiments, the bone formation marker is measured in a serum or plasma sample from the subject.

In some embodiments, the method comprises selecting the subject on the basis of the subject's alkaline phosphatase level (e.g., the subject's baseline level of alkaline phosphatase prior to the start of treatment with the NGF antagonist, or a change in the subject's level of alkaline phosphatase from baseline to a timepoint after the start of treatment with the NGF antagonist). In some embodiments, the method comprises determining whether the subject is a candidate for continued treatment with the NGF antagonist (e.g., an anti-NGF antibody or antigen-binding fragment thereof) by comparing the level of alkaline phosphatase in the subject at a timepoint after the start of treatment to a baseline level of alkaline phosphatase in the subject prior to or at the start of treatment, wherein a subject is identified as a candidate for continued treatment if the subject does not have an increase in the level of alkaline phosphatase that is above a threshold value.

In some embodiments, the subject's level of bone formation marker (e.g., alkaline phosphatase) is measured at a timepoint that is about 8 weeks to about 16 weeks from the start of treatment with the NGF antagonist, e.g., about 8 weeks from the start of treatment with the NGF antagonist, about 9 weeks, about 10 weeks, about 11 weeks, about 12 weeks, about 13 weeks, about 14 weeks, about 15 weeks, or about 16 weeks.

In some embodiments, a subject's level of bone formation marker (e.g., alkaline phosphatase) is measured at more than one timepoint after the start of treatment with the NGF antagonist, e.g., to determine whether a patient being treated with the NGF antagonist is a suitable candidate for continued treatment. In some embodiments, the subject's level of bone formation marker (e.g., alkaline phosphatase) is determined at baseline, and then at a first timepoint after the start of treatment with the NGF antagonist (e.g., at a first timepoint that is about 8 weeks to about 16 weeks from the start of treatment with the NGF antagonist), and then at a second timepoint that is subsequent to the first timepoint (e.g., at a second timepoint that is at least about 8 weeks, 10 weeks, 12 weeks, 14 weeks, 16 weeks, 18 weeks, 20 weeks, 24 weeks, 28 weeks, 32 weeks, 36 weeks, 40 weeks, 44 weeks, 48 weeks, 52 weeks, or more after the first timepoint, or about 8 weeks to about 16 weeks after the first timepoint). In some embodiments, the level of bone formation marker (e.g., alkaline phosphatase) at each timepoint following the start of treatment (e.g., at the first timepoint, second timepoint, and any subsequent timepoint) is compared to the baseline level of marker in the subject, wherein the subject is identified as a suitable candidate for continued treatment with NGF antagonist if the subject's marker level at the specified timepoint (e.g., first timepoint, second timepoint, etc.) is not increased above a threshold value, relative to a baseline level of the bone formation marker in the subject prior to or at the start of treatment.

In some embodiments, the threshold value is a 5, 6, 7, 8, 9, 10, 11, 12, 13, 14, 15, 16, 17, 18, 19, or 20-point increase from baseline. In some embodiments, a “point” is measured in U/L (e.g., a 20-point increase is equivalent to an increase of 20 U/L). In some embodiments, the threshold value is a 20-point increase in the level of alkaline phosphatase, relative to the baseline level of alkaline phosphatase. In some embodiments, the threshold value is a 15-point increase in the level of alkaline phosphatase, relative to the baseline level of alkaline phosphatase. In some embodiments, the threshold value is a 10-point increase in the level of alkaline phosphatase, relative to the baseline level of alkaline phosphatase. In some embodiments, the subject's baseline level of alkaline phosphatase is an average of two or more measurements of alkaline phosphatase for the subject, wherein each measurement is taken prior to or at the start of treatment.

In some embodiments, the alkaline phosphatase is serum alkaline phosphatase. In some embodiments, the alkaline phosphatase is bone-specific alkaline phosphatase. In some embodiments, the alkaline phosphatase is measured using an enzymatic assay. In some embodiments, the alkaline phosphatase is measured using an immunoassay (e.g., ELISA). Assays for measuring alkaline phosphatase are known in the art. See, e.g., Tang et al., TrAC Trends in Analytical Chemistry, 2019, 113:32-43; and Roudsari and Mahjoub, Caspian J Intern Med, 2012, 3:478-483. Assays for measuring alkaline phosphatase are also commercially available. See, e.g., Abcam Colorimetric Alkaline Phosphatase Assay Kit (ab83369), Abcam, Cambridge, UK; Ostase® BAP EIA, Immunodiagnostic Systems, Tyne & Wear, UK;

In some embodiments, a subject is identified as a candidate for treatment with an NGF antagonist, or as a candate for continued treatment with an NGF antagonist, on the basis of a combination of factors disclosed herein (e.g., a combination of the number of large joints that exhibit osteoarthritis at baseline; lack of pre-existing joint arthropathy or history of joint arthropathy; and having a change in bone formation marker (e.g., alkaline phosphatase) level relative to baseline that is not more than a threshold value).

Methods of Treating Subjects with Pain Due to Osteoarthritis of the Knee and/or Hip

In one aspect, disclosed herein are methods of treating or reducing pain associated with osteoarthritis in a subject. In some embodiments, the subject has chronic osteoarthritis of the knee and/or hip.

In some embodiments, the subject is a subject who has been identified as a suitable candidate for treatment with an NGF antagonist, e.g., according to the methods disclosed above. For example, in some embodiments, the subject to be treated has 1-3 joints (e.g., 1-2 joints, or no more than 2 large joints) that exhibit osteoarthritis, wherein the large joints are selected from the group consisting of knee joint, hip joint, and shoulder joint.

In some embodiments, the methods comprise treating a subject who is resistant, non-responsive, or inadequately responsive to treatment with a standard analgesic, or a subject who has an intolerance to standard analgesic therapy, including paracetamol/acetaminophen, oral NSAIDs, and opioid therapy. This subset of patients represents a patient population with an unmet medical need for pain relief therapy. These patients may benefit from treatment with an NGF antagonist such as fasinumab, which has the potential to be both safe and effective in this difficult to treat patient population.

In some embodiments, the methods comprise treating a subject with an initial dose of an NGF antagonist, or an initial course of treatment with an NGF antagonist (e.g., one or more doses of an anti-NGF antibody or antigen-binding fragment thereof as described herein), then determining whether the subject is a candidate for continued treatment with the NGF antagonist. In some embodiments, the step of determining whether the subject is a candidate for continued treatment comprises comparing the level of a bone formation marker (e.g., alkaline phosphatase) in the subject at a timepoint after the start of treatment with the NGF antagonist to a baseline level of the marker (e.g., alkaline phosphatase) in the subject prior to or at the start of treatment, wherein a subject is identified as a candidate for continued treatment if the subject does not have an increase in the level of the marker (e.g., alkaline phosphatase) that is above a threshold value. In some embodiments, the threshold value is a 5, 6, 7, 8, 9, 10, 11, 12, 13, 14, 15, 16, 17, 18, 19, or 20-point increase from baseline. In some embodiments, the threshold value is a 15-point increase from baseline. In some embodiments, the threshold value is a 10-point increase from baseline. In some embodiments, the timepoint is about 8 weeks to about 16 weeks from the start of treatment with the NGF antagonist.

In some embodiments, the step of determining whether the subject is a candidate for continued treatment comprises comparing the level of the bone formation marker (e.g., alkaline phosphatase) in the subject at two or more timepoints after the start of treatment. In some embodiments, the method comprises treating a subject with an initial dose of an NGF antagonist, or an initial course of treatment with an NGF antagonist (e.g., one or more doses of an anti-NGF antibody or antigen-binding fragment thereof as described herein), then determining whether the subject is a candidate for continued treatment by evaluating the level of alkaline phosphatase in the subject at a first timepoint after the initial dose or initial course of treatment relative to the subject's baseline level of alkaline phosphatase, then treating the subject with a secondary dose of the NGF antagonist or a secondary course of treatment with the NGF antagonist, then determining whether the subject is a candidate for continued treatment by evaluating the level of alkaline phosphatase in the subject at a second timepoint after the administration of the secondary dose or secondary course of treatment relative to the subject's baseline level of alkaline phosphatase. In some embodiments, the first timepoint is about 8 weeks to about 16 weeks from the start of the initial dose or initial course of treatment. In some embodiments, the second timepoint is about 8 weeks to about 16 weeks from the start of the secondary dose or secondary course of treatment. In some embodiments, the second timepoint is more than 16 weeks from the start of the secondary dose or secondary course of treatment.

In some embodiments, the step of determining whether the subject is a candidate for continued treatment comprises measuring one or more pain-associated parameters. In some embodiments, the step of determining whether the subject is a candidate for continued treatment comprises measuring the level of pain in the subject and identifying the subject as a candidate for continued treatment if the subject exhibits a decrease in pain at a timepoint after the start of treatment relative to a baseline level of pain in the subject prior to or at the start of treatment.

Examples of “pain-associated parameters” include: (a) Western Ontario and McMaster Universities Osteoarthritis Index (WOMAC) pain score; (b) WOMAC physical function subscale scores; (c) Patient Global Assessment (PGA) score; (d) the knee and/or hip pain Numerical Rating Scale (NRS) score; (e) the short form health survey (SF-36) subscale scores; and (f) the EuroQoL 5 Dimensions 5 Level Questionnaire (EQ-5D-5L).

In some embodiments, the level of pain is measured using:
(a) the Western Ontario and McMaster Universities Osteoarthritis Index (WOMAC) pain subscale score;
(b) the Numeric Rating Scale (NRS) for joint pain; and/or
(c) Patient Global Assessment (PGA) of knee and/or hip pain.

As used herein, the term “baseline,” with regard to a pain-associated parameter, means the value of the pain-associated parameter for a subject or group of subjects prior to or at the start of treatment with the NGF antagonist. To determine whether a pain-associated parameter has “improved,” the parameter is quantified at baseline and at one or more time points after administration of the pharmaceutical composition disclosed herein. For example, a pain-associated parameter may be measured at various time points after administration of the anti-NGF antibody, e.g., at day 1, day 2, day 3, day 4, day 5, day 6, day 7, day 8, day 9, day 12, day 18, day 22, day 36, day 50, day 57, day 64, day 78, day 85, day 92, day 106, day 113, day 120; or at the end of week 1, week 2, week 3, week 4, week 5, week 6, week 7, week 8, week 9, week 10, week 11, week 12, week 13, week 14, week 15, week 16, week 24, week 36, week 44, week 52, week 72, week 104, or longer, after the initial treatment with the NGF antagonist. The difference between the value of the parameter at a particular time point following initiation of treatment and the value of the parameter at baseline is used to establish whether there has been an “improvement” (e.g., a decrease) in the pain associated parameter. In some embodiments, the methods disclosed herein result in an improvement from baseline of at least about 10%, 20%, 30%, 40%, 50%, or more in one or more pain-associated parameter following administration of the NGF antagonist, e.g., an anti-NGF antibody such as fasinumab.

In one aspect, treatment according to the methods disclosed herein results in an improvement in one or more of: (a) Western Ontario and McMaster Universities Osteoarthritis Index (WOMAC) pain score; (b) WOMAC physical function subscale scores; (c) Patient Global Assessment (PGA) score; (d) Numeric Rating Scale (NRS) of the average walking index for joint pain; (e) the EuroQoL 5 Dimensions 5 Level Questionnaire; (f) the 36-item Short Form Survey (SF-36); (g) the Healthcare Resource Utilization Questionnaire; (h) the Work Productivity and Activity Impairment Osteoarthritis; or (i) the Treatment Satisfaction Questionnaire for Medication.

In some embodiments, the pain-associated parameter is the WOMAC pain subscale score. The WOMAC pain subscale score is a composite index of 5 questions related to joint pain while walking, using stairs, at rest in bed, sitting or lying, and standing and is described in Bellamy N. WOMAC Osteoarthritis Index: A User's Guide. London, Ontario, Canada: Victoria Hospital; 1995. In some embodiments, individual WOMAC questions are scored on a scale of 0-10. The scores from each of the 5 questions are averaged. In some embodiments, the subject is identified as a candidate for continued treatment if the subject exhibits at least 10%, at least 20%, at least 30%, or at least 40% improvement in WOMAC pain subscale score, relative to the baseline level, at a specified timepoint (e.g., at least 4 weeks or at least 8 weeks after the start of treatment, or about 8 weeks to 16 weeks from the start of treatment, or 12 weeks, 16 weeks, or more after the start of treatment).

In some embodiments, the pain-associated parameter is the WOMAC physical function subscale score. The WOMAC physical function subscale score measures 17 items for functional limitation (scale of 0-10). Physical functioning questions cover everyday activities such as stair use, standing up from a sitting or lying position, standing, bending, walking, getting in and out of a car, shopping, putting on or taking off socks, lying in bed, getting in or out of a bath, sitting, and heavy and light household duties. In some embodiments, the subject is identified as a candidate for continued treatment if the subject exhibits at least 10%, at least 20%, at least 30%, or at least 40% improvement in WOMAC physical function subscale score, relative to the baseline level, at a specified timepoint (e.g., at least 4 weeks or at least 8 weeks after the start of treatment, or about 8 weeks to 16 weeks from the start of treatment, or 12 weeks, 16 weeks, or more after the start of treatment).

In some embodiments, the pain-associated parameter is the Patient Global Assessment (PGA) score. The Patient Global Assessment of OA is a patient-rated assessment of patient current disease state on a 5-point Likert scale (1=very well; 2=well; 3=fair; 4=poor; and 5=very poor). In some embodiments, the subject is identified as a candidate for continued treatment if the subject exhibits at least 10%, at least 20%, at least 30%, or at least 40% improvement in PGA score, relative to the baseline level, at a specified timepoint (e.g., at least 4 weeks or at least 8 weeks after the start of treatment, or about 8 weeks to 16 weeks from the start of treatment, or 12 weeks, 16 weeks, or more after the start of treatment).

In some embodiments, the pain-associated parameter is the Numeric Rating Scale (NRS) average walking index joint pain. This NRS score is a patient-rated assessment of index joint pain, in which the patient indicates their average daily index joint pain over the past 24 hours or weekly index joint pain. In some embodiments, the subject is identified as a candidate for continued treatment if the subject exhibits at least 10%, at least 20%, at least 30%, or at least 40% improvement in NRS score, relative to the baseline level, at a specified timepoint (e.g., at least 4 weeks or at least 8 weeks after the start of treatment, or about 8 weeks to 16 weeks from the start of treatment, or 12 weeks, 16 weeks, or more after the start of treatment).

### Osteoarthritis Polygenic Risk Score

In some embodiments, the methods of mitigating risk of joint destruction, methods for identifying or selecting a subject having OA who is a suitable candidate for treatment with an NGF antagonist, and methods of treating subjects having pain associated with OA of the knee or hip in a subject further comprise selecting the subject on the basis of an osteoarthritis polygenic risk score (OA-PRS). In some embodiments, the subject is selected on the basis of having an OA-PRS that is less than a threshold OA-PRS, wherein the OA-PRS comprises a weighted aggregate of a plurality of genetic variants associated with osteoarthritis.

Polygenic risk scores (PRSs) combine information from a large number of genetic variants derived from disease association studies to create a single composite quantitative measure for each individual which reflects their genetically-derived disease risk.

Risk assessments using large numbers of genetic variants offers the advantage of increased predictive power. In some embodiments, one or more of the genetic variants is a single nucleotide polymorphism (SNP). In some embodiments, one or more of the genetic variants is an insertion. In some embodiments, one or more of the genetic variants is a deletion. In some embodiments, one or more of the genetic variants is a structural variant. In some embodiments, one or more of the genetic variants is a copy-number variation.

In some embodiments, an OA-PRS is determined for a subject by identifying whether one or more genetic variants associated with a risk of developing severe OA requiring joint replacement or developing AA are present in a biological sample from the subject and calculating an OA-PRS for the subject based on the identified genetic variants, wherein the OA-PRS is calculated by aggregating, such as by summing, the risk score (or weighted risk score) associated with each identified genetic variant. The number of identified genetic variants can be at least about 2 genetic variants, at least about 5 genetic variants, at least about 10 genetic variants, at least about 15 genetic variants, at least about 20 genetic variants, at least about 30 genetic variants, at least about 40 genetic variants, at least about 50 genetic variants, at least about 95 genetic variants, at least about 100 genetic variants, at least about 200 genetic variants, at least about 500 genetic variants, at least about 1000 genetic variants, at least about genetic variants, at least about 25,000 genetic variants, at least about 50,000 genetic variants, at least about 100,000 genetic variants, at least about 250,000 genetic variants, at least about 500,000 genetic variants, at least about 750,000 genetic variants, at least about 1,000,000 genetic variants, at least about 5,000,000 genetic variants, or at least about genetic variants associated with a risk of developing severe OA requiring joint replacement or developing AA.

As an exemplary method, an OA-PRS can be determined from, for example, data obtained from a genome-wide association study (GWAS) of disease risk. For example, in a representative hypothetical GWAS, a GWAS may have identified four genetic variants associated with a disease. Each of the genetic variants may be associated with one or more genes. A value, such as an Odds Ratio, can be calculated for each individual genetic variant. A particular subject's OA-PRS can be determined by multiplying the log value of the individual Odds Ratio for each variant by the Number Effect Alleles (which is the number of copies of the genetic variant in the genome; i.e., either 0, 1, or 2), and then summing the resultant values to obtain a total score. Thus, the subject's OA-PRS is the sum of the individual values taking into consideration any number of genetic variants associated with the particular disease, phenotype, biomarker, laboratory measure, or clinical endpoint. In this example, the OA-PRS is a weighted score because each genetic variant may carry a different weight depending on the particular Odds Ratio and the Number Effect Alleles value.

### NGF Antagonists

According to certain exemplary embodiments of the present disclosure, a subject in need thereof is administered an NGF antagonist (e.g., a pharmaceutical composition comprising an NGF antagonist). In some embodiments, an NGF antagonist is an anti-NGF antibody or antigen-binding fragment thereof. In certain exemplary embodiments, the NGF antagonist is an anti-NGF antibody, or antigen-binding fragment thereof comprising a heavy chain variable region (HCVR), light chain variable region (LCVR), and/or complementarity determining regions (CDRs) comprising any of the amino acid sequences of the anti-NGF antibodies as set forth in any of International Publication No. WO 2018/102294, U.S. Pat. No. 7,988,967, and U.S. Patent Application Publication No. 2012/0097565.

In some embodiments, the anti-NGF antibody or antigen-binding fragment thereof comprises the three heavy chain complementarity determining regions (HCDR1, HCDR2, and HCDR3) of a heavy chain variable region (HCVR) comprising the amino acid sequence of SEQ ID NO:2, and the three light chain complementarity determining regions (LCDR1, LCDR2, and LCDR3) of a light chain variable region (LCVR) comprising the amino acid sequence of SEQ ID NO:10. In some embodiments, the anti-NGF antibody or antigen-binding fragment thereof comprises an HCDR1 comprising the amino acid sequence of SEQ ID NO:4, an HCDR2 comprising the amino acid sequence of SEQ ID NO:6, an HCDR3 comprising the amino acid sequence of SEQ ID NO:8, an LCDR1 comprising the amino acid sequence of SEQ ID NO:12, an LCDR2 comprising the amino acid sequence AAF (SEQ ID NO:14), and an LCDR3 comprising the amino acid sequence of SEQ ID NO:16.

In some embodiments, the anti-NGF antibody or antigen-binding fragment thereof comprises an HCVR comprising the amino acid sequence of SEQ ID NO:2 and an LCVR comprising the amino acid sequence of SEQ ID NO:10. In some embodiments, the anti-NGF antibody or antigen-binding fragment thereof comprises a heavy chain comprising the amino acid sequence of SEQ ID NO:17 and/or a light chain comprising the amino acid sequence of SEQ ID NO:18.

In some embodiments of the present disclosure, the antibody or antigen-binding fragment thereof comprises an HCVR/LCVR amino acid sequence pair consisting of SEQ ID NOs: 2/10. In some embodiments, the antibody or antigen-binding fragment thereof comprises a heavy chain and/or light chain comprising variants of any of the HCVR, LCVR, and/or CDR amino acid sequences disclosed herein having one or more conservative substitutions. For example, the present disclosure includes the use of anti-NGF antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein. In some embodiments, an anti-NGF antibody comprises 10 or fewer (e.g., no more than 8, no more than 6, no more than 5, no more than 4, no more than 3, no more than 2, or no more than 1) conservative amino acid substitutions relative to an HCVR or LCVR amino acid sequence disclosed herein, and no more than one conservative amino acid substitution within any of the CDR amino acid sequences disclosed herein.

In some embodiments, the NGF antagonist is fasinumab. Fasinumab is a fully-human high-affinity monoclonal antibody directed against NGF (see U.S. Pat. No. 7,988,967 and PCT Publication No. WO 2009/023540 and WHO Drug Information Vol. 26, No. 2, (2012), which are all hereby incorporated by reference in their entirety). The amino acid sequences of the heavy chain and light chain variable regions and the CDRs portions as well as the nucleotide sequences of fasinumab are described in Table 3. Nucleic and amino acid sequences corresponding to the indicated SEQ ID NOs can be found in Table 1A and 1B. As used herein, “fasinumab” also includes bioequivalents of fasinumab. The term “bioequivalent,” as used herein with reference to fasinumab, refers to anti-NGF antibodies or NGF-binding proteins or fragments thereof that are pharmaceutical equivalents or pharmaceutical alternatives whose rate and/or extent of absorption do not show a significant difference with that of fasinumab when administered at the same molar dose under similar experimental conditions, either single dose or multiple dose. In some embodiments, the term refers to antigen-binding proteins that bind to NGF which do not have clinically meaningful differences with fasinumab in their safety, purity and/or potency.

TABLE 1A

| | AMINO ACID SEQ ID NOs: | | | | | | | |
|---|---|---|---|---|---|---|---|---|
| Antibody | | HCDR | HCDR | HCDR | | LCDR | LCDR | LCDR |
| Designation | HCVR | 1 | 2 | 3 | LCVR | 1 | 2 | 3 |
| Fasinumab | 2 | 4 | 6 | 8 | 10 | 12 | 14 | 16 |

TABLE 1B

| | NUCLEIC ACID SEQ ID NOs: | | | | | | | |
|---|---|---|---|---|---|---|---|---|
| Antibody | | HCDR | HCDR | HCDR | | LCDR | LCDR | LCDR |
| Designation | HCVR | 1 | 2 | 3 | LCVR | 1 | 2 | 3 |
| Fasinumab | 1 | 3 | 5 | 7 | 9 | 11 | 13 | 15 |

In some embodiments, the NGF antagonist is an antibody disclosed in U.S. Pat. Nos. 7,601,818, 7,795,413, 8,106,167, or U.S. Pat. No. 8,198,410. In some embodiments, the NGF antagonist is fulranumab.

In some embodiments, the NGF antagonist is an antibody disclosed in U.S. Pat. Nos. 7,449,616, 7,659,364, 8,088,384, 8,540,990, 9,708,398, or U.S. Pat. No. 10,188,600. In some embodiments, the NGF antagonist is tanezumab.

The instant disclosure encompasses antibodies having one or more mutations in the hinge, C_{H}2 or C_{H}3 region which may be desirable, for example, in production, to improve the yield of the desired antibody form.

### Preparation of Human Antibodies

Methods for generating human antibodies in transgenic mice are known in the art. Any such known methods can be used in the context of the present disclosure to make human antibodies that specifically bind to human NGF.

Using VELOCIMMUNE™ technology (see, for example, U.S. Pat. No. 6,596,541, Regeneron Pharmaceuticals) or any other known method for generating monoclonal antibodies, high affinity chimeric antibodies to NGF are initially isolated having a human variable region and a mouse constant region. The VELOCIMMUNE® technology involves generation of a transgenic mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibody are isolated and operably linked to DNA encoding the human heavy and light chain constant regions. The DNA is then expressed in a cell capable of expressing the fully human antibody.

Generally, a VELOCIMMUNE® mouse is challenged with the antigen of interest, and lymphatic cells (such as B-cells) are recovered from the mice that express antibodies. The lymphatic cells may be fused with a myeloma cell line to prepare immortal hybridoma cell lines, and such hybridoma cell lines are screened and selected to identify hybridoma cell lines that produce antibodies specific to the antigen of interest. DNA encoding the variable regions of the heavy chain and light chain may be isolated and linked to desirable isotypic constant regions of the heavy chain and light chain. Such an antibody protein may be produced in a cell, such as a CHO cell. Alternatively, DNA encoding the antigen-specific chimeric antibodies or the variable domains of the light and heavy chains may be isolated directly from antigen-specific lymphocytes.

Initially, high affinity chimeric antibodies are isolated having a human variable region and a mouse constant region. The antibodies are characterized and selected for desirable characteristics, including affinity, selectivity, epitope, etc, using standard procedures known to those skilled in the art. The mouse constant regions are replaced with a desired human constant region to generate the fully human antibody of the disclosure, for example wild-type or modified IgG1 or IgG4. While the constant region selected may vary according to specific use, high affinity antigen-binding and target specificity characteristics reside in the variable region.

In general, the antibodies that can be used in the methods of the present disclosure possess high affinities, as described above, when measured by binding to antigen either immobilized on solid phase or in solution phase. The mouse constant regions are replaced with desired human constant regions to generate the fully human antibodies of the disclosure. While the constant region selected may vary according to specific use, high affinity antigen-binding and target specificity characteristics reside in the variable region.

In one embodiment, a human antibody or antigen-binding fragment thereof that specifically binds NGF and that can be used in the methods disclosed herein comprises the three heavy chain CDRs (HCDR1, HCDR2, and HCDR3) contained within a heavy chain variable region (HCVR) having the amino acid sequence of SEQ ID NO: 2, and the three light chain CDRs (LCDR1, LCDR2, and LCDR3) contained within a light chain variable region (LCVR) having the amino acid sequence of SEQ ID NO: 10. Methods and techniques for identifying CDRs within HCVR and LCVR amino acid sequences are well known in the art and can be used to identify CDRs within the specified HCVR and/or LCVR amino acid sequences disclosed herein. Exemplary conventions that can be used to identify the boundaries of CDRs include, e.g., the Kabat definition, the Chothia definition, and the AbM definition. In general terms, the Kabat definition is based on sequence variability, the Chothia definition is based on the location of the structural loop regions, and the AbM definition is a compromise between the Kabat and Chothia approaches. See, e.g., Kabat, “Sequences of Proteins of Immunological Interest,” National Institutes of Health, Bethesda, Md. (1991); Al-Lazikani et al., J. Mol. 273:927-948 (1997); and Martin et al., Proc. Natl. Acad. Sci. USA 86:9268-9272 (1989). Public databases are also available for identifying CDR sequences within an antibody.

### Pharmaceutical Compositions

In one aspect, the present disclosure provides methods that comprise administering an NGF antagonist to a patient, wherein the NGF antagonist (e.g., an anti-NGF antibody or antigen-binding fragment thereof) is contained within a pharmaceutical composition that comprises one or more pharmaceutically acceptable vehicle, carriers, and/or excipients. Various pharmaceutically acceptable carriers and excipients are well-known in the art. See, e.g., Remington's Pharmaceutical Sciences, Mack Publishing Company, Easton, PA. In some embodiments, the carrier is suitable for intravenous, intramuscular, oral, intraperitoneal, intrathecal, transdermal, topical, or subcutaneous administration.

In some some embodiments, the pharmaceutical composition comprises an injectable preparation, such as a dosage form for intravenous, subcutaneous, intracutaneous and intramuscular injections, drip infusions, etc. These injectable preparations may be prepared by known methods. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared can be filled in an appropriate ampoule.

The dose of active agent (e.g., antibody) administered to a patient according to the methods of the present disclosure may vary depending upon the age and the size of the patient, symptoms, conditions, route of administration, and the like. The dose is typically calculated according to body weight or body surface area. Depending on the severity of the condition, the frequency and the duration of the treatment can be adjusted. Effective dosages and schedules for administering pharmaceutical compositions comprising anti-NGF antibodies may be determined empirically; for example, patient progress can be monitored by periodic assessment, and the dose adjusted accordingly. Moreover, interspecies scaling of dosages can be performed using well-known methods in the art (e.g., Mordenti et al., 1991, Pharmaceut. Res. 8:1351). Specific exemplary doses of anti-NGF antibodies, and administration regimens involving the same, that can be used in the context of the present disclosure are disclosed elsewhere herein.

Various delivery systems are known and can be used to administer the pharmaceutical composition, e.g., encapsulation in liposomes, microparticles, microcapsules, recombinant cells capable of expressing the mutant viruses, receptor mediated endocytosis (see, e.g., Wu et al., 1987, J. Biol. Chem. 262:4429-4432). Methods of administration include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. In some embodiments, a pharmaceutical composition as disclosed herein is administered intravenously. In some embodiments, a pharmaceutical composition as disclosed herein is administered subcutaneously.

In some embodiments, a pharmaceutical composition of the present disclosure is contained within a container. Thus, in another aspect, containers comprising a pharmaceutical composition as disclosed herein are provided. For example, in some embodiments, a pharmaceutical composition is contained within a container selected from the group consisting of a glass vial, a syringe, a pen delivery device, and an autoinjector.

In some embodiments, a pharmaceutical composition of the present disclosure is delivered, e.g., subcutaneously or intravenously, with a standard needle and syringe. In some embodiments, the syringe is a pre-filled syringe. In some embodiments, a pen delivery device or autoinjector is used to deliver a pharmaceutical composition of the present disclosure (e.g., for subcutaneous delivery). A pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Examples of suitable pen and autoinjector delivery devices include, but are not limited to AUTOPEN™ (Owen Mumford, Inc., Woodstock, UK), DISETRONIC™ pen (Disetronic Medical Systems, Bergdorf, Switzerland), HUMALOG MIX 75/25™ pen, HUMALOG™ pen, HUMALIN 70/30™ pen (Eli Lilly and Co., Indianapolis, IN), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, NJ), OPTIPEN™, OPTIPEN PRO™ OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present disclosure include, but are not limited to the SOLOSTAR™ pen (sanofi-aventis), the FLEXPEN™ (Novo Nordisk), and the KWIKPEN™ (Eli Lilly), the SURECLICK™ Autoinjector (Amgen, Thousand Oaks, CA), the PENLET™ (Haselmeier, Stuttgart, Germany), the EPIPEN (Dey, L. P.), and the HUMIRA™ Pen (Abbott Labs, Abbott Park Ill.).

In some embodiments, the pharmaceutical composition is delivered using a controlled release system. In one embodiment, a pump may be used (see Langer, supra; Sefton, 1987, CRC Crit. Ref. Biomed. Eng. 14:201). In another embodiment, polymeric materials can be used; see, Medical Applications of Controlled Release, Langer and Wise (eds.), 1974, CRC Pres., Boca Raton, Florida. In yet another embodiment, a controlled release system can be placed in proximity of the composition's target, thus requiring only a fraction of the systemic dose (see, e.g., Goodson, 1984, in Medical Applications of Controlled Release, supra, vol. 2, pp. 115-138). Other controlled release systems are discussed in the review by Langer, 1990, Science 249:1527-1533.

In some embodiments, pharmaceutical compositions for use as described herein are prepared into dosage forms in a unit dose suited to fit a dose of the active ingredient(s). Such dosage forms in a unit dose include, for example, tablets, pills, capsules, injections (ampoules), suppositories, etc.

Exemplary pharmaceutical compositions comprising an anti-NGF antibody that can be used in the context of the present disclosure are disclosed, e.g., in U.S. Patent Application Publication No. US 2012/0014968.

### Dosage and Administration

Typically, an amount of NGF antagonist (e.g., anti-NGF antibody or antigen-binding fragment thereof as disclosed herein) that is administered to a subject according to the methods of the present disclosure is a therapeutically effective amount. As used herein, the phrase “therapeutically effective amount” means an amount of NGF antagonist that results in one or more of: (a) an improvement in one or more pain-associated parameters (as defined elsewhere herein); and/or (b) a detectable improvement in one or more symptoms or indicia of pain. A “therapeutically effective amount” also includes an amount of NGF antagonist that inhibits, prevents, lessens, or delays the progression of pain in a subject. In some embodiments, the NGF antagonist is an anti-NGF antibody, e.g., an antibody comprising three heavy chain complementarity determining region (HCDR) sequences (HCDR1, HCDR2, HCDR3) comprising SEQ ID NOs: 4, 6 and 8, respectively, and three light chain complementarity determining (LCDR) sequences (LCDR1, LCDR2, LCDR3) comprising SEQ ID NOs: 12, 14 and 16, respectively. In some embodiments, the anti-NGF antibody is fasinumab.

In the case of an anti-NGF antibody or antigen-binding fragment thereof, a therapeutically effective amount can be from about 0.05 mg to about 600 mg, e.g., about 0.05 mg, about 0.1 mg, about 0.5 mg, about 1.0 mg, about 1.5 mg, about 2.0 mg, about 2.5 mg, about 3.0 mg, about 4.0 mg, about 5.0 mg, about 6.0 mg, about 7.0 mg, about 8.0 mg, about 9.0 mg, about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 510 mg, about 520 mg, about 530 mg, about 540 mg, about 550 mg, about 560 mg, about 570 mg, about 580 mg, about 590 mg, or about 600 mg, of the anti-NGF antibody. In some embodiments, a therapeutically effective amount can be from about 1 mg to about 10 mg of an anti-NGF antibody, or from about 0.5 mg to about 5 mg of an anti-NGF antibody. In some embodiments, about 1 mg, about 3 mg, about 6 mg, or about 9 mg of an anti-NGF antibody is administered to a subject. In some embodiments, about 1 mg of an anti-NGF antibody is administered to a subject.

The amount of NGF antagonist contained within the individual doses may be expressed in terms of milligrams of antibody per kilogram of patient body weight (i.e., mg/kg). For example, the NGF antagonist may be administered to a patient at a dose of about 0.0001 to about 10 mg/kg of patient body weight. For example, the NGF antagonist may be administered to a patient at a dose of about 0.03 to about 3 mg/kg of patient body weight. For example, the NGF antagonist may be administered to a patient at a dose of about 0.03 to about 3 mg/kg of patient body weight.

In some embodiments, an NGF antagonist or pharmaceutical composition comprising an NGF antagonist is administered to a subject at a dosing frequency of about four times a week, twice a week, once a week, once every two weeks, once every three weeks, once every four weeks, once every five weeks, once every six weeks, once every eight weeks, once every twelve weeks, or less frequently so long as a therapeutic response is achieved. In certain embodiments involving the administration of a pharmaceutical composition comprising an anti-NGF antibody, such as fasinumab, once every 4 weeks (Q4W) dosing at an amount of about mg to about 10 mg, e.g., about 1 mg, can be employed. In certain embodiments involving the administration of a pharmaceutical composition comprising an anti-NGF antibody, such as fasinumab, once every 8 weeks (Q8W) dosing at an amount of about 0.5 mg to about 10 mg, e.g., about 1 mg, can be employed.

In some embodiments, multiple doses of an NGF antagonist may be administered to a subject over a defined time course. In some embodiments, the methods of the present disclosure comprise sequentially administering to a subject multiple doses of an NGF antagonist. As used herein, “sequentially administering” means that each dose of NGF antagonist is administered to the subject at a different point in time, e.g., on different days separated by a predetermined interval (e.g., hours, days, weeks or months). The present disclosure includes methods which comprise sequentially administering to the patient a single initial dose of an NGF antagonist, followed by one or more secondary doses of the NGF antagonist, and optionally followed by one or more tertiary doses of the NGF antagonist.

The terms “initial dose,” “secondary dose(s),” and “tertiary dose(s)” refer to the temporal sequence of administration of the NGF antagonist. Thus, the “initial dose” is the dose which is administered at the beginning of the treatment regimen; the “secondary doses” are the doses which are administered after the initial dose; and the “tertiary doses” are the doses which are administered after the secondary doses. The initial, secondary, and tertiary doses may all contain the same amount of NGF antagonist, but generally may differ from one another in terms of frequency of administration. In certain embodiments, however, the amount of NGF antagonist contained in the initial, secondary and/or tertiary doses varies from one another (e.g., adjusted up or down as appropriate) during the course of treatment.

In some embodiments, each initial, secondary, and/or tertiary dose is administered 1 to 14 (e.g., 1, 1%, 2, 2%, 3, 3%, 4, 4%, 5, 5%, 6, 6%, 7, 7%, 8, 8%, 9, 9%, 10, 10%, 11, 11%, 12, 12%, 13, 13%, 14, 14%, or more) weeks after the immediately preceding dose. The phrase “the immediately preceding dose,” as used herein, means, in a sequence of multiple administrations, the dose of NGF antagonist which is administered to a patient prior to the administration of the very next dose in the sequence with no intervening doses.

The methods of the disclosure may comprise administering to a patient any number of initial, secondary, and/or tertiary doses of an NGF antagonist. For example, in certain embodiments, only a single initial dose is administered to the patient. In other embodiments, two or more (e.g., 2, 3, 4, 5, 6, 7, 8, or more) initial doses are administered to the patient. As another example, in certain embodiments, only a single secondary dose is administered to the patient. In other embodiments, two or more (e.g., 2, 3, 4, 5, 6, 7, 8, or more) secondary doses are administered to the patient. Likewise, in certain embodiments, only a single tertiary dose is administered to the patient. In other embodiments, two or more (e.g., 2, 3, 4, 5, 6, 7, 8, or more) tertiary doses are administered to the patient.

In some embodiments, the methods of the disclosure comprise administering an anti-NGF antibody or antigen-binding fragment thereof comprising an HCDR1, HCDR2, HCDR3, LCDR1, LCDR2, and LCDR3 comprising the amino acid sequences of SEQ ID NOs: 4, 6, 8, 12, 14, and 16, respectively, at a dose of about 1.0 mg. In some embodiments, the anti-NGF antibody comprising an HCDR1, HCDR2, HCDR3, LCDR1, LCDR2, and LCDR3 comprising the amino acid sequences of SEQ ID NOs: 4, 6, 8, 12, 14, and 16, respectively, is administered at a dose of about 1.0 mg about every 4 weeks (Q4W). In some embodiments, the anti-NGF antibody comprising an HCDR1, HCDR2, HCDR3, LCDR1, LCDR2, and LCDR3 comprising the amino acid sequences of SEQ ID NOs: 4, 6, 8, 12, 14, and 16, respectively, is administered at a dose of about 1.0 mg about every 8 weeks (Q8W).

In some embodiments, the methods of the disclosure comprise administering an anti-NGF antibody comprising an HCVR/LCVR amino acid sequence pair of SEQ ID NOs: 2/10 at a dose of about 1.0 mg. In some embodiments, the anti-NGF antibody comprising an HCVR/LCVR amino acid sequence pair of SEQ ID NOs: 2/10 is administered at a dose of about 1.0 mg about every 4 weeks (Q4W). In some embodiments, the anti-NGF antibody comprising an HCVR/LCVR amino acid sequence pair of SEQ ID NOs: 2/10 is administered at a dose of about 1.0 mg about every 8 weeks (Q8W).

### Combination Therapies

The methods of the present disclosure, according to certain embodiments, comprise administering to the subject one or more additional therapeutic agents in combination with the NGF antagonist. As used herein, the expression “in combination with” means that the additional therapeutic agents are administered before, after, or concurrent with the pharmaceutical composition comprising the NGF antagonist. The term “in combination with” also includes sequential or concomitant administration of NGF antagonist and a second therapeutic agent.

For example, when administered “before” the pharmaceutical composition comprising the NGF antagonist, the additional therapeutic agent may be administered about 72 hours, about 60 hours, about 48 hours, about 36 hours, about 24 hours, about 12 hours, about 10 hours, about 8 hours, about 6 hours, about 4 hours, about 2 hours, about 1 hour, about 30 minutes, about 15 minutes or about 10 minutes prior to the administration of the pharmaceutical composition comprising the NGF antagonist. When administered “after” the pharmaceutical composition comprising the NGF antagonist, the additional therapeutic agent may be administered about 10 minutes, about 15 minutes, about 30 minutes, about 1 hour, about 2 hours, about 4 hours, about 6 hours, about 8 hours, about 10 hours, about 12 hours, about 24 hours, about 36 hours, about 48 hours, about 60 hours or about 72 hours after the administration of the pharmaceutical composition comprising the NGF antagonist.

Administration “concurrent” or with the pharmaceutical composition comprising the NGF antagonist means that the additional therapeutic agent is administered to the subject in a separate dosage form within less than 5 minutes (before, after, or at the same time) of administration of the pharmaceutical composition comprising the NGF antagonist, or administered to the subject as a single combined dosage formulation comprising both the additional therapeutic agent and the NGF antagonist.

The additional therapeutic agent may be, e.g., another NGF antagonist (e.g. see the NGF antibodies described in U.S. Pat. No. 7,449,616 (tanezumab); U.S. Pat. Nos. 7,569,364; 7,655,232; 8,088,384; WO2011049758 (fulranumab)), an IL-1 antagonist (including, e.g., an IL-1 antagonist as set forth in U.S. Pat. No. 6,927,044), an IL-6 antagonist, an IL-6R antagonist (including, e.g., an anti-IL-6R antibody as set forth in U.S. Pat. No. 7,582,298), an opioid, acetaminophen, a local anesthestic, an NMDA modulator, a cannabinoid receptor agonist, a P2X family modulator, a VR1 antagonist, a substance P antagonist, a Na v 1.7 antagonist, a cytokine or cytokine receptor antagonist, an antiepileptic drug, a steroid, other inflammatory inhibitors such as inhibitors of caspase-1, p38, IKK1/2, CTLA-4lg and a corticosteroid.

In one embodiment, the additional therapeutic agent is not a NSAID. In some embodiments, the additional therapeutic agent excludes NSAIDs.

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1: Clinical Studies Evaluating the Efficacy of Fasinumab in Reducing Pain or Improving Physical Function Versus Placebo or NSAID Treatment in Patients with Osteoarthritis of the Knee or Hip

The efficacy and safety of fasinumab in patients with moderate-to-severe knee and/or hip OA pain has been studied in multiple clinical trials, including NCT02447276 (R475-PN-1227), NCT02683239 (R475-PN-1523), NCT03161093 (R475-OA-1611), and NCT03304379 (R475-OA-1688). The clinical trial design and results for NCT02447276 are described in Dakin et al., Arthritis Rheumatol 2019, 71:1824-1834, incorporated by reference herein. The clinical trial design and results for NCT02683239, NCT03161093, and NCT03304379 are described below.

NCT02683239 (R475-PN-1523, or “FACT LTS”), NCT03161093 (R475-OA-1611, or “FACT OA1”), and NCT03304379 (R475-OA-1688, or “FACT OA2”) each were Phase 3 trials evaluating fasinumab in adults with moderate-to-severe chronic pain associated with osteoarthritis (OA) of the knee or hip. All three of these trials enrolled patients with OA of the knee or hip who had inadequate pain relief with acetaminophen and were intolerant to or had inadequate pain relief with opioids, or were unwilling or unable to take opioids. Complete inclusion and exclusion criteria for the trials are described at clinicaltrials.gov/ct2/show/NCT03161093, clinicaltrials.gov/ct2/show/NCT03304379, and clinicaltrials.gov/ct2/show/NCT02683239.

FACT OA1 and FACT OA2 assessed the efficacy of fasinumab in comparison to nonsteroidal anti-inflammatory drugs (NSAIDs; naproxen, diclofenac, or celecoxib) for treatment of OA. FACT OA1 includes results from a total of 1,801 patients randomized to one of four treatment arms: fasinumab 1 mg SC once every 8 weeks (Q8W) (n=304), fasinumab 1 mg SC once every 4 weeks (Q4W) (n=639), naproxen 500 mg twice daily (n=644), or placebo (n=214). In FACT OA2, patients were randomized into one of six treatment groups: fasinumab 1 mg SC once every 4 weeks (Q4W), fasinumab 3 mg SC once every 4 weeks (Q4W), fasinumab 6 mg SC once every 8 weeks (Q8W), celecoxib 200 mg once daily, diclofenac 75 mg twice daily, or placebo. While the FACT OA2 trial was still ongoing, enrollment into the fasinumab 3 mg Q4W and 6 mg Q8W groups was stopped for safety considerations; patients enrolled in those dose regimens discontinued study drug and entered the post-treatment follow-up period.

Both FACT OA1 and FACT OA2 achieved their co-primary endpoints for fasinumab 1 mg Q4W, showing that patients treated with fasinumab 1 mg Q4W experienced significantly reduced pain and improved physical function compared to placebo (p<0.01 for all endpoints). Fasinumab patients in both trials also reported improvements in their pain and physical function (WOMAC score) compared to those treated with the highest-approved prescription doses of NSAIDS. A summary of the Q4W results from these two trials is shown in Table 2.

TABLE 2
Efficacy results from FACT OA1 and FACT OA2 trials for
fasinumab 1 mg Q4W dosing (change from baseline)

| | |
|---|---|
| FACT OA1 Trial | FACT OA2 Trial |
| (at Week 16) | (at Week 24) |

COMPARISON WITH PLACEBO (CO-PRIMARY ENDPOINTS)

| | | Fasinumab | | | Fasinumab | |
|---|---|---|---|---|---|---|
| | Placebo | 1 mg Q4W | Difference | Placebo | 1 mg Q4W | Difference |
| | N = 214 | N = 639 | vs. placebo | N = 264 | N = 524 | vs. Placebo |
| WOMAC | −26% | −39% | −0.66 | −30% | −42% | −0.79 |
| pain | (−1.82) | (−2.48) | p = 0.0003 | (−1.99) | (−2.79) | nominal |
| | | | | | | p < 0.0001 |
| WOMAC | −25% | −39% | −0.67 | −25% | −39% | −0.82 |
| physical | (−1.71) | (−2.41) | p = 0.0001 | (−1.81) | (−2.63) | nominal |
| function | | | | | | p < 0.0001 |

COMPARISON WITH NSAIDS (SECONDARY ENDPOINTS)

| | | | | Diclofenac | | |
|---|---|---|---|---|---|---|
| | | | | 2 × 75 mg/day | | |
| | Naproxen | | | or | | Difference |
| | 2 × 500 mg/ | Fasinumab | Difference | celecoxib | Fasinumab | vs. |
| | day | 1 mg/month | vs. | 200 mg/d | 1 mg/month | diclofenac/ |
| | N = 644 | N = 639 | naproxen | N = 525 | N = 524 | celecoxib |
| WOMAC | −33% | −39% | −0.35 | −37% | −42% | −0.30 |
| pain | (−2.13) | (−2.48) | nominal | (−2.49) | (−2.79) | nominal |
| | | | p = 0.0050 | | | p = 0.0503 |
| WOMAC | −31% | −39% | −0.41 | −34% | −39% | −0.36 |
| physical | (−1.98) | (−2.41) | nominal | (−2.27) | (−2.63) | nominal |
| function | | | p = 0.0006 | | | p = 0.0152 |

The FACT LTS study evaluated the long-term safety and tolerability of fasinumab, including adverse events of special interest such as joint damage (e.g. arthropathies), compared with placebo. A total of 2,411 patients were randomized to one of the following treatment arms: (1) fasinumab 1 mg subcutaneous (SC) every 8 weeks (Q8W); (2) fasinumab 1 mg SC every 4 weeks (Q4W); or (3) matching placebo SC Q4W. This study also included a substudy evaluating the efficacy of fasinumab in these same patient groups (n=215 in fasinumab 1 mg Q8W, n=217 in fasinumab 1 mg Q4W, n=214 in placebo). The results of the substudy achieved both co-primary endpoints, showing that patients in each fasinumab treatment group experienced significantly reduced pain and improved physical function (WOMAC score) compared to placebo.

Across all three of the fasinumab Phase 3 trials, higher rates of arthropathies were adjudicated in patients treated with fasinumab compared with placebo or NSAIDs, which were dose-dependent.

### Example 2: Identifying Patients at Higher Risk for Joint Related Adverse Events

The 1 mg dose of fasinumab, administered every 4 weeks or every 8 weeks, has demonstrated efficacy as described above in Example 1, with a likely advantage over the best possible efficacy that could be expected with NSAIDS. To improve risk-benefit for patients, analyses were conducted to identify populations of patients that might be at greater risk for joint-related adverse events. An integrated summary of safety (ISS) analysis was performed using pooled data from the clinical studies R475-PN-1612 (NCT03285646), R475-PN-1227 (NCT02447276), R475-PN-1523 (NCT02683239), R475-OA-1611 (NCT03161093), R475-OA-1688 (NCT03304379), R475-OA-1758 (NCT03691974), and MT-5547-J01 (NCT03245008).

This example presents three analyses that describe populations with differing levels of risk. Two analyses are grounded in baseline patient characteristics and one is based on early (8-16 weeks) post-treatment change to a widely available biomarker, alkaline phosphatase. Risk-mitigation strategies based on these analyses would allow for both discontinuation of some high-risk patients before damage is detected on imaging and not treating other high-risk patients from the start.

As used in this Example, the term “adjudicated arthropathy” or “AA” is an umbrella term that encompasses the following conditions: rapidly progressive OA type 1 or 2, subchondral insufficiency fractures, and primary osteonecrosis. The term “destructive arthropathy” or “DA” refers to abnormal bone fragmentation, destruction, or fracture over a short period of time, which are observed by X-ray radiography and which are features inconsistent with radiographic findings typically observed in conventional advanced OA. The term “joint replacement” or “JR” refers to joint replacement surgery.

Baseline Burden of Large-Joint OA Identifies Subgroups of Patients with Lower and Higher Risk for AA/DA/JR

To examine different phenotypes of OA (generalized OA in particular) a subgroup analysis based on the number of large joints (knees, hips, shoulders) with OA at baseline was performed. OA joints were defined as any hip or knee with baseline KL 2 score or above and any shoulder with a score of mild, moderate or severe. Thus, the minimum number of joints with OA was 1 and the maximum number could be 6. Three subgroups were arbitrarily designated 1-2, 3-4, and 5-6 large joints with OA at baseline.

The analyses are based on large numbers of patients: 2103 patients in the placebo group, 1676 patients in the fasinumab 1 mg Q8W group (also referred to herein as “1Q8”), and 3009 patients in the 1 mg Q4W group (also referred to herein as “1Q4”). The percentage of the population in each subgroup was similar across treatment arms; the 1-2 joint subgroup ranged from 55.8 to 59.5% of the entire population, the range for the 3-4 joint subgroup was 33.6 to 37.4%, and the range for in the 5-6 joint subgroup was 6.7 to 7.1%.

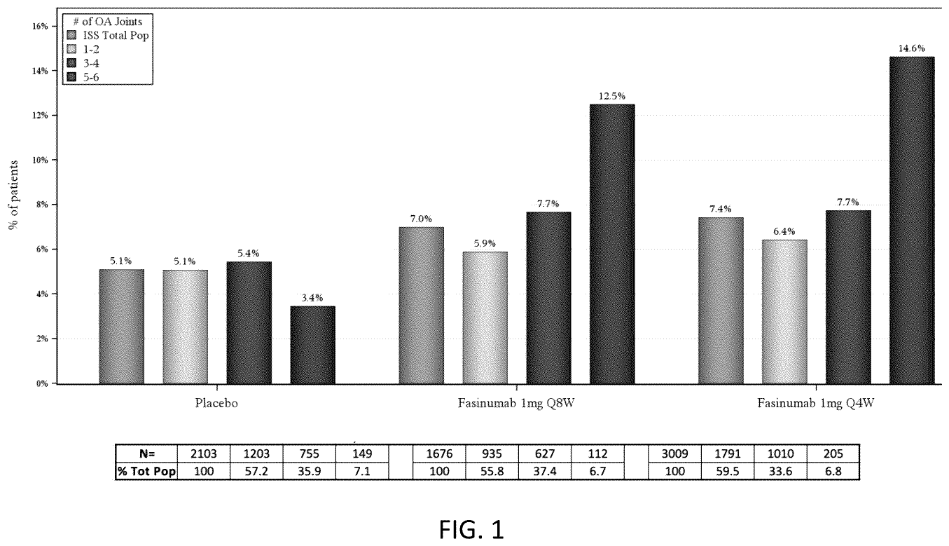

Joint replacements are the most unambiguous and clinically relevant safety endpoint. FIG. 1 shows the percentage of patients who had joint replacement in the total ISS population versus the OA joint subgroups by treatment arm. There was no clear trend between subgroups in the placebo arm; however, in both treatment arms, increasing number of OA joints at baseline was associated with increased percentage of patients undergoing JR. For the 1Q4 arm, 14.6% of patients in the 5-6 joint subgroup underwent JR versus 6.4% in the 1-2 joint subgroup. For the 1Q8 arm, 12.5% of patients in the 5-6 joint subgroup underwent JR versus 5.9% in the 1-2 joint subgroup. In the 1-2 joint subgroup, the difference from placebo was 1.3% for the 1Q4 arm and 0.8% for the 1Q8 arm. In the treatment arms, the JR rates appear to increase almost exponentially from the 1-2 joint subgroup to the 5-6 joint subgroup suggesting that the observation is influenced more by an underlying phenotype rather than the number of joints at risk for JR.

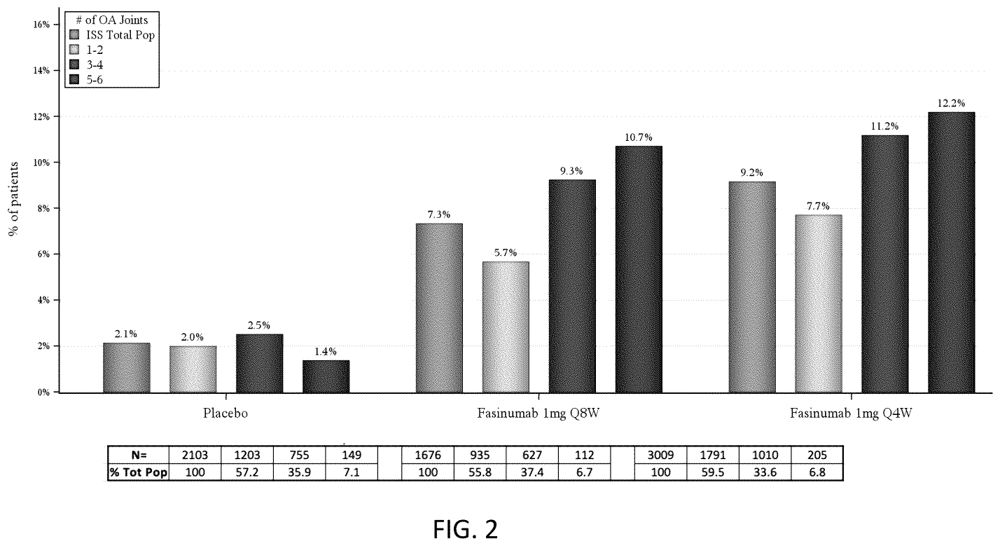

FIG. 2 shows the percentage of patients who had AAs in the total ISS population versus the OA joint subgroups by treatment arm. There was no clear trend between subgroups in the placebo arm; however; in both treatment arms, increasing number of OA joints at baseline was associated with increased percentage of patients who develop AA. For the 1Q4 arm, 12.2% of patients in the 5-6 joint subgroup developed AA versus 7.7% in the 1-2 joint subgroup. For the 1Q8 arm, 10.7% of patients in the 5-6 joint subgroup developed AA versus 5.7% in the 1-2 joint subgroup. The difference in all AA from placebo was 5.7% for the 1Q4 arm and 3.7% for the 1Q8 arm.

The majority (— 70-80%) of AA events were RPOA-1. For the 1-2 joint subgroup, the 1Q4 arm had an RPOA-1 rate of 5.8% versus 1.7% for placebo, a difference of 4.1%. Also, for the 1-2 joint subgroup, the 1Q8 arm had an RPOA-1 rate of 4.8% versus 1.7% for placebo, a difference of 3.1%.

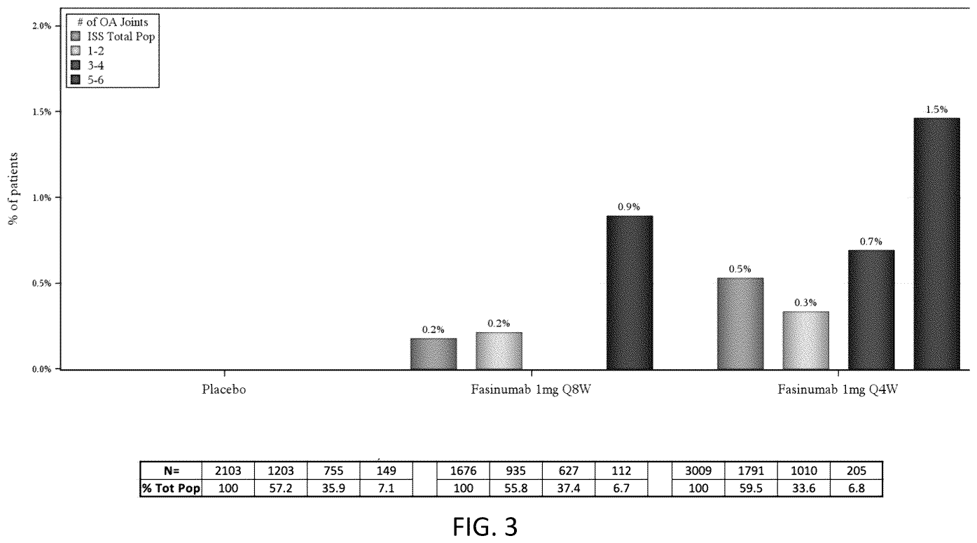

DAs are the most clinically relevant of the AAs. FIG. 3 shows the percentage of patients who had DAs in the total ISS population versus the OA joint subgroups by treatment arm. There were no DAs in the placebo arm. In both treatment arms, increasing number of OA joints at baseline were associated with increased percentage of patients who develop DA. For the 1Q4 arm, 1.5% of patients in the 5-6 joint subgroup underwent JR versus 0.3% in the 1-2 joint subgroup. For the 1Q8 arm, 0.9% of patients in the 5-6 joint subgroup underwent JR versus 0.2% in the 1-2 joint subgroup. The rates of DA in the fasinumab arms are in line with reported estimates for increased yearly rates of the more serious adverse effects of NSAIDs: non-fatal MI and cardiovascular death (CNT trialists 2013); and well below what would be expected for patients with cardiovascular risk factors taking NSAIDs (Olsen 2012).

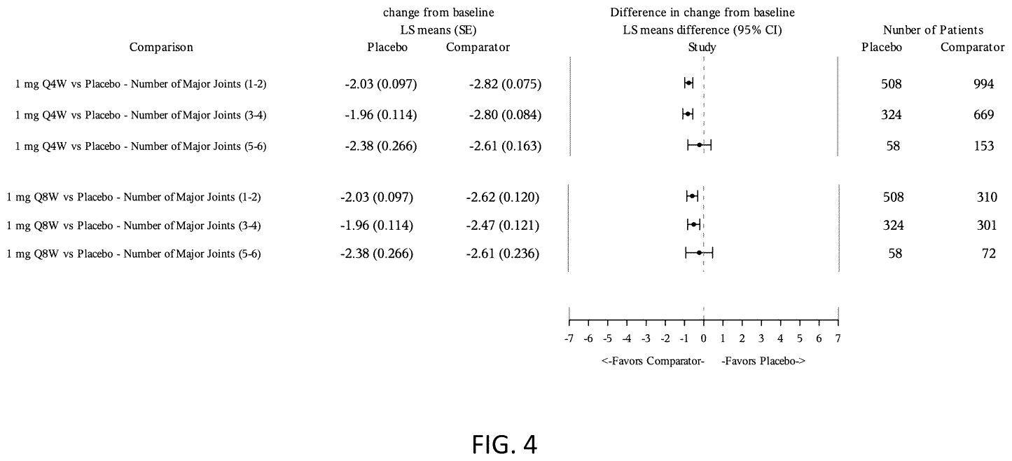

The integrated summary of efficacy (ISE) examined the change in WOMAC pain subscale score at 16 weeks in each of the above subgroups and is illustrated in the Forrest plot below (FIG. 4). All subgroups showed numerically greater improvement compared with placebo; however, the 95% confidence intervals excluded zero only for the 1-2 and 3-4 joint subgroups. This was observed for both the 1Q4 regimen and the 1Q8 regimen. Therefore, the subgroup with highest risk of joint related adverse events was also the subgroup showed the least benefit.

In summary, this analysis demonstrates that a higher baseline burden of osteoarthritis is associated with increased risk for joint related adverse events in OA patients treated with fasinumab.

Change from Baseline in Serum Alkaline Phosphatase Identifies Subgroups of Patients with Higher Odds of DA

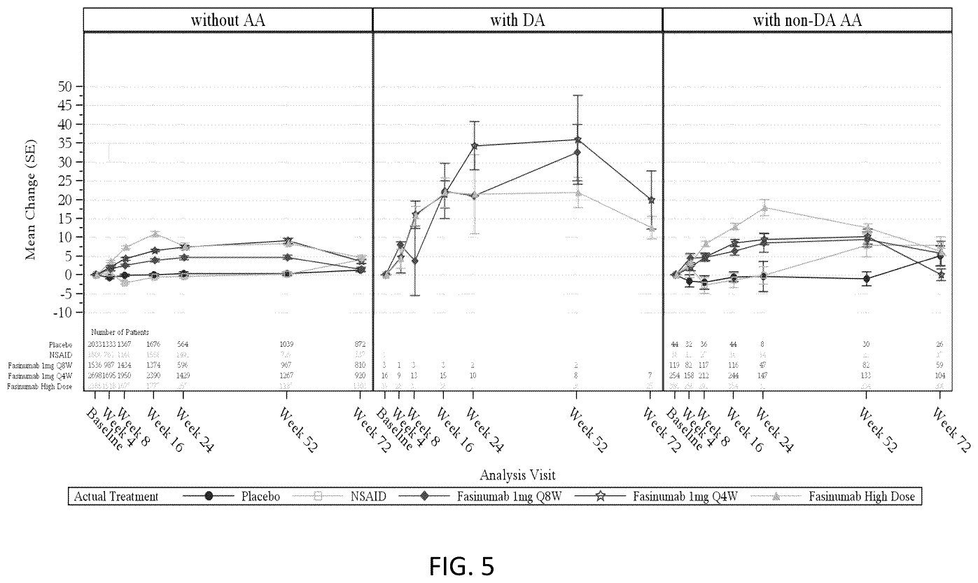

It has been previously observed that, following initiation of therapy with fasinumab, there is 5 to 10-point(U/L) mean increase in alkaline phosphatase (depending upon dose) that peaks by approximately 16 weeks (Dakin et al., Arthritis Rheumatol 2019, 71:1824-1834). These changes are well within the normal range, but the magnitude of change is dose related and returns toward baseline following treatment discontinuation, indicating an association with fasinumab. The ISS provides a useful dataset to explore the relationship of alkaline phosphatase to AAs because low frequency events, such as DAs, can be pooled. FIG. 5 shows the change from baseline in alkaline phosphatase by dose in the ISS dataset. Three non-overlapping subgroups are shown: patients without AA, patients with DA and patients with non-DA AAs.

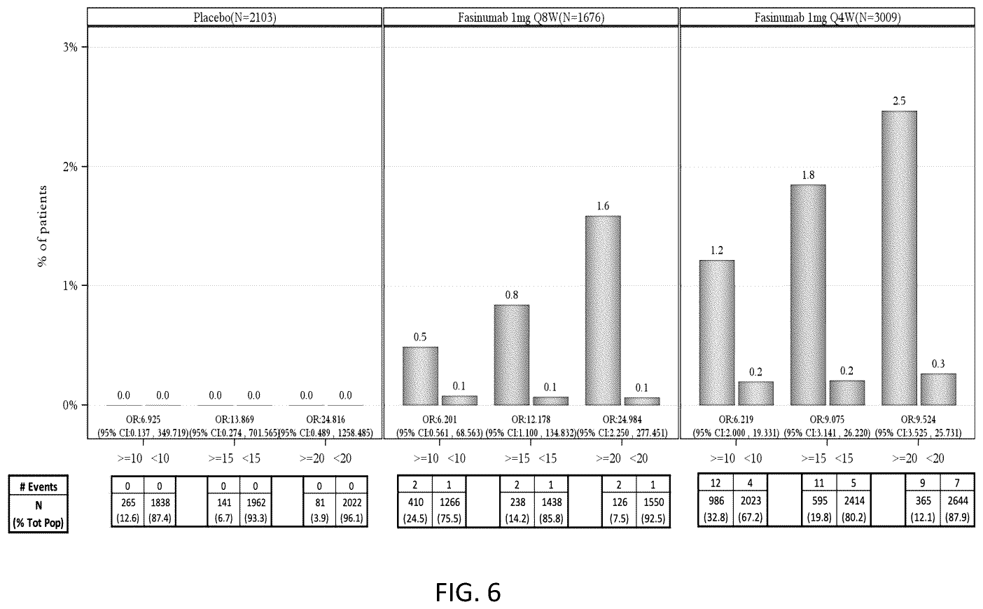

DA events are clearly associated with a larger change from baseline in alkaline phosphatase when compared with both the patients who did not develop DA/AA and also the patients who had other sub-types of AA. For DA patients at all doses, the mean change from baseline in alkaline phosphatase was 20 points (U/L) by 16 weeks. These changes are observed much earlier than the median time for detection of DA (284 days for 1 mg Q8W; 343.5 days for 1 mg Q4W; and 364.5 days for the pooled high dose that includes 3 mg Q4W, 6 mg Q4W, 9 mg Q8W, and 9 mg Q4W), raising the possibility that early changes in alkaline phosphatase could be used to predict later DA events. To explore this possibility, three different cut-offs at 16 weeks were chosen (10, 15 and 20 U/L change from baseline) and the rate of DA above and below each cut-off was examined (FIG. 6). Of note, the standard deviation for change in alkaline phosphatase in the placebo arm is 12.9 at 8 weeks and 12.7 at 16 weeks.

There were no DA events in the placebo arm. The patients whose change from baseline in alkaline phosphatase was above each designated cut-off at 16 weeks showed higher odds of DA, with odd ratios ranging from approximately 6 to 25. 95% confidence intervals for the odds ratios excluded 1 for all 1Q4 cutoffs and the 20-point cutoff for 1Q8. In the 1 mg Q4W arm, 2.5% of patients with 20-point change from baseline at 16 weeks developed DA versus 0.3% of patients with <20-point change. In the 1 mg Q8W arm, 1.6% of patients with 20-point change from baseline at 16 weeks developed DA versus 0.1% of patients with <20-point change. At the lower cut-offs, more true positives were identified; however, larger number of false positives were also identified.

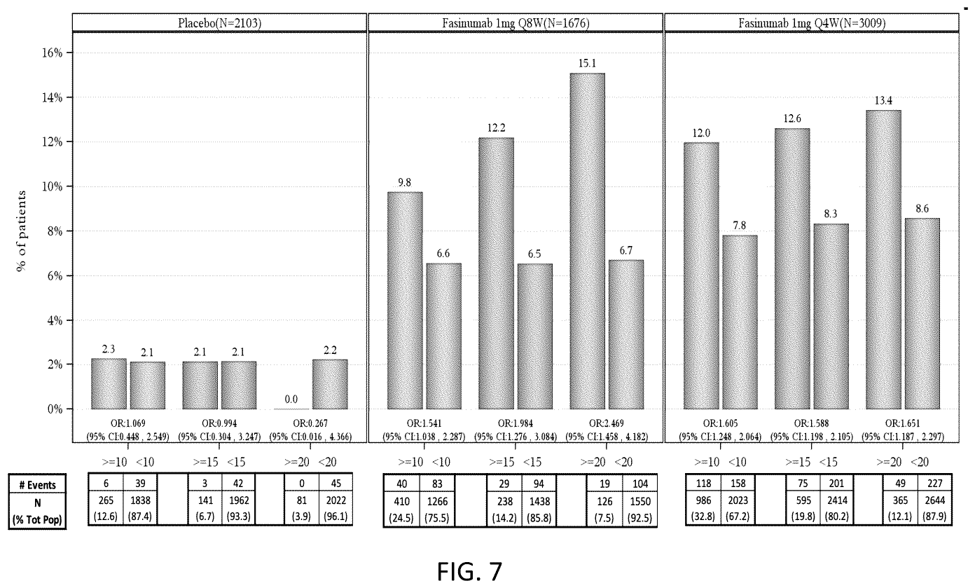

A similar analysis was performed over all AAs (70-80% RPOA-1) which also shows higher odds for AA in patients above each of the designated cut-off at 16 weeks (FIG. 7). Odds ratios ranged from approximately 1.5 to 2.5 with all 95% confidence intervals excluding 1. AA events were detected in the placebo arm, however, change of alkaline phosphatase did not have any impact on the odds of placebo patients developing AA overall. These data suggest that there is a fasinumab-specific process playing a role in AA development that may be predicted by early changes in this widely available biomarker. It is understood that use of alkaline phosphatase to identify patients at higher risk of DA/AA may result in identification of a large number of patients who would benefit from drug without an event (false positives); however, the identification of a group with improved safety risk may be a desirable trade-off.

### Baseline Burden of OA and Change in Alkaline Phosphatase are Complementary for Risk Reduction

Both the baseline burden of OA and the change in alkaline phosphatase identify patients with higher risk of AA/DA/JR; these findings are observed only in the fasinumab arms, not the placebo arm, indicating an NGF-specific mechanism. The results raise the possibility of excluding high-risk patients and treating only patients with lower risk of joint-related adverse events, using more than one approach. To understand whether there is an interaction between the two approaches, and to explore a number of potential risk mitigation options, a post-hoc analysis was performed on the ISS data set to examine the impact of alkaline phosphatase within various joint subgroups. Because efficacy was preserved in the 1-2 and 3-4 joint subgroups as discussed above, the rates of AA/DA/JR were examined in overlapping subgroups: patients with 1-2, 1-3 and 1-4 joints with OA at baseline. For each of these subgroups, five potential cut-offs for change in alkaline phosphatase were explored: no change, 5-, 10-, 15- and 20-point change from baseline.

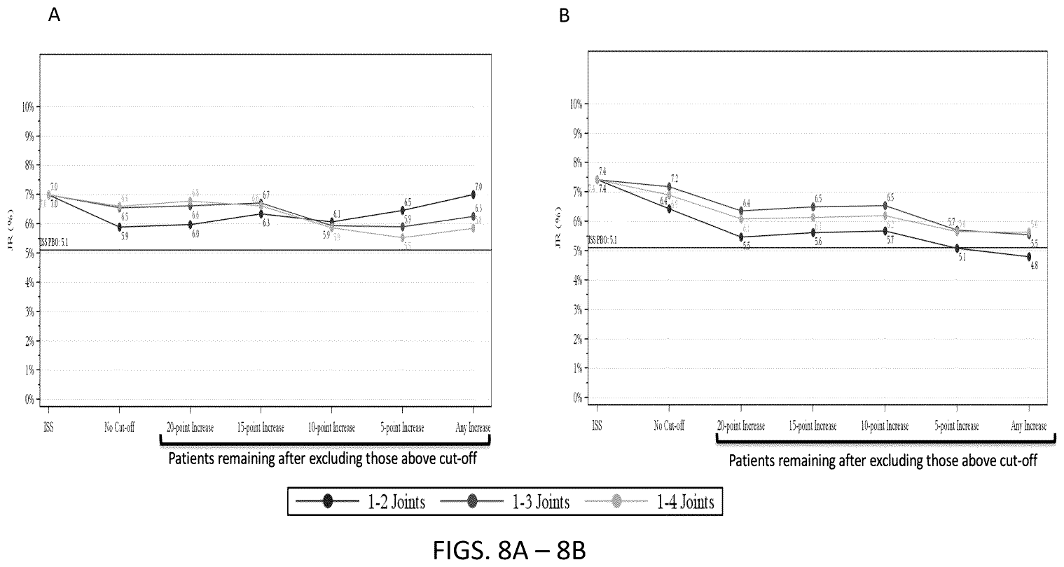

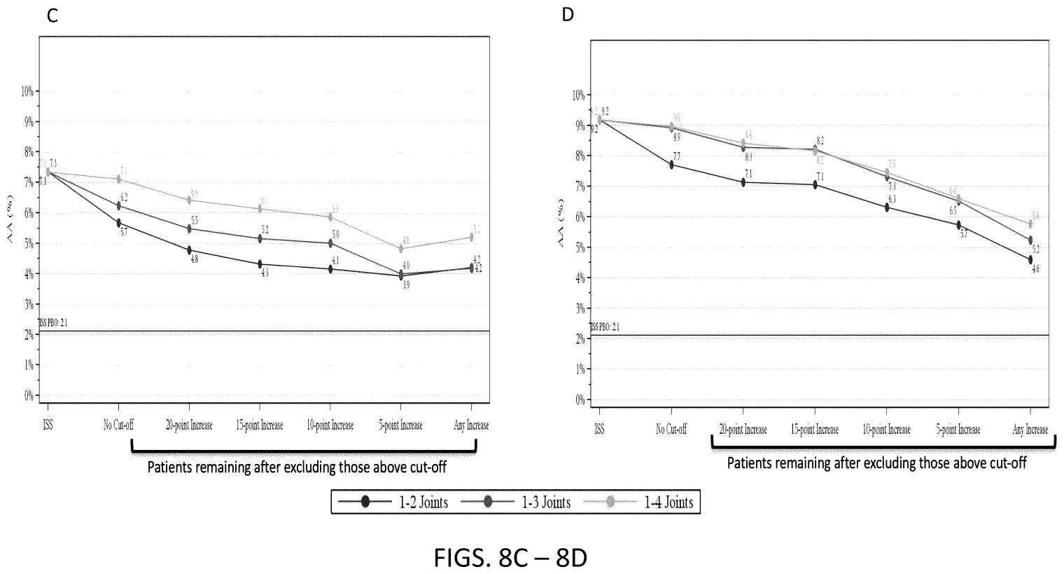

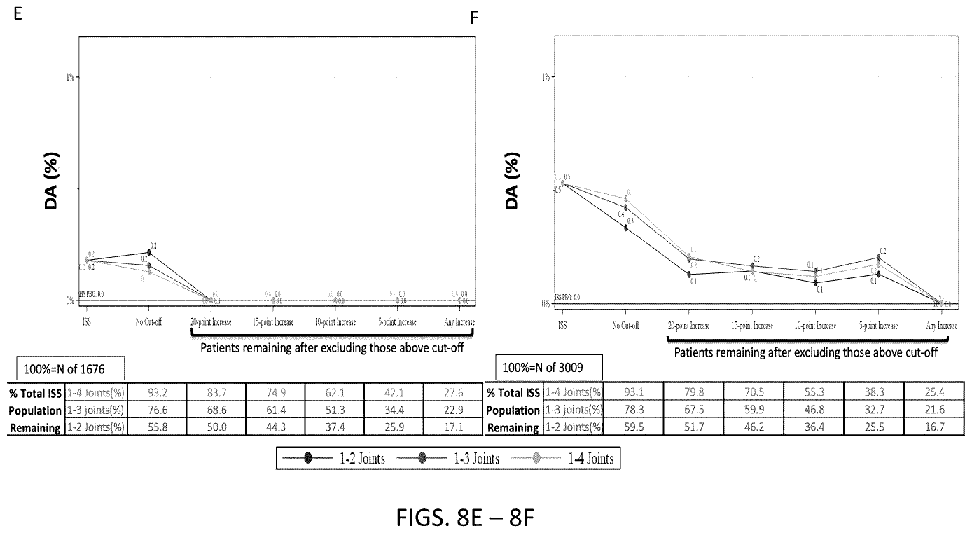

FIGS. 8A-8D show the percentage of patients with AA/DA/JR following exclusion of those who have a change in alkaline phosphatase at or above the designated cut-off at any time between weeks 8 and 16. In each of FIGS. 8A-8D, the X-axis represents overlapping bins of patients that become successively smaller as one looks left to right (percentage of the total population remaining in each bin is shown in the table beneath the graph). The left-most bin (ISS) shows the AA/DA/JR rate for the total ISS population. The next bin to the right (No Cut-off) shows the AA/DA/JR rate 1-2, 1-3 and 1-4 joint subgroups. The next 5 bins to the right show the rates of AA/DA/JR in the patients who remain after excluding those above the designated cut-off for change in alkaline phosphatase. The placebo rate from the total ISS population is expressed as a solid black line.

For each of the joint related adverse events, the burden of OA and change in alkaline phosphatase are complementary from the perspective of risk-reduction. FIGS. 8A, 8C, and 8E show the reduction of risk of JR, AA, and DA, respectively, in patients treated with 1 mg fasinumab Q8W, for patients with different OA burden at different cut-off values for change in alkaline phosphatase. Similarly, FIGS. 8B, 8D, and 8F show the reduction of risk of JR, AA, and DA, respectively, in patients treated with 1 mg fasinumab Q4W, for patients with different OA burden at different cut-off values for change in alkaline phosphatase. For example, for the 1Q4 treatment arm and in the 1-2 joint subgroup, the JR rate is reduced from 7.4% in the total ISS population, to 6.4% in the 1-2 joint subgroup, to 5.5% in the population below the 20-point alkaline phosphatase cut-off—a difference of 0.4% from the ISS placebo rate (FIG. 8B). Similarly, for the 1Q4 treatment arm and in the 1-2 joint subgroup, the overall AA rate (70-80% RPDA-1) is reduced from 9.2% in the total ISS population, to 7.7% in the 1-2 joint subgroup, to 7.1% in the population below the 20-point alkaline phosphatase cut-off—a difference of 5.6% from the ISS placebo rate (FIG. 8D). Last, the DA rate is reduced from 0.5% in the total ISS population, to 0.3% in the 1-2 joint subgroup, to 0.1% in the population below the 20-point alkaline phosphatase (FIG. 8F).

Furthermore, lower cut-offs for change in alkaline phosphatase were associated with lower rates of each joint-related adverse event. For example, for the 1Q4 treatment arm and in the 1-2 joint subgroup, the rates of JR and DA could be reduced to approximately the placebo rates. See, FIGS. 8B and 8F. For overall AAs (70-80% RPOA-1), could be reduced to 4.6%, a difference of 2.5% from the placebo rate. See, FIG. 8D.

These findings suggest that identification of lower risk populations using both the baseline burden of OA and early change in alkaline phosphatase would be complementary, lowering risk beyond what would be achievable with either strategy alone. Further, risk can be reduced to approach the placebo rate for the most clinically relevant outcomes JR and DA: for the 1 mg Q4W arm, a difference of 0.4% for JR and 0.1% for DA (for the alkaline phosphatase cut-off of 20). These rates are similar to or below the reported increased risk for more serious AE's associated with NASIDS such as cardiovascular death, non-fatal MI and GI bleed (CNT trialists 2013).

### CONCLUSION

In patients with OA of the knee and/or hip, the risk of AA, DA, and JR can be reduced by the use of specific patient selection criteria. First, the identification and selection of patients with no more than 4 large joints with OA at baseline (e.g., patients with 1-4 large joints with OA, 1-3 large joints with OA, or 1-2 large joints with OA) can reduce the risk of AA, DA, and JR. Second, by monitoring patients' alkaline phosphatase levels and discontinuing fasinumab treatment in patients with increases in alkaline phosphatase at or above specified cutoff values after an initial treatment period (e.g., 8-16 weeks of treatment), patients with lower risk of AA, DA, and JR can be selected for continued treatment. Moreover, these selection strategies can be used in combination to further reduce the risk of AA, DA, and JR in patients with OA for whom fasinumab may be efficacious.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions.

Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

TABLE 3
Nucleic acid and amino acid sequences

| SEQ ID | | |
|---|---|---|
| NO | Description | Sequence |
| 1 | Fasinumab HCVR, | CAGGTGCAGCTGGTGCAGTCTGGGGCTGAGGTGAAGAAGCCTGGGGCCTCAGTGAA |
| | nucleic acid | GGTCTCCTGCAAGGTTTCCGGATTCACCCTCACTGAATTATCCATTCACTGGGTGCG |
| | sequence | ACAGGCTCCTGGAAAAGGGCTTGAGTGGATGGGAGGTTTTGATCCTGAAGATGGTG |
| | | AAACAATCTACGCACAGAAGTTCCAGGGCAGAGTCACCATGACCGAGGACACATCT |
| | | ACAGACACAGCCTACATGGAGCTGACCAGCCTGAGATCGGAAGACACGGCCGTGT |
| | | ATTACTGTTCAACGATTTTTGGAGTGGTTACCAACTTTGACAACTGGGGCCAGGGAA |
| | | CCCTGGTCACCGTCTCCTCA |
| 2 | Fasinumab HCVR, | QVQLVQSGAEVKKPGASVKVSCKVSGFTLTELSIHWVRQAPGKGLEWMGGFDPEDGE |
| | amino acid | TIYAQKFQGRVTMTEDTSTDTAYMELTSLRSEDTAVYYCSTIFGVVTNFDNWGQGTL |
| | sequence | VTVSS |
| 3 | Fasinumab HCDR1, | GGATTCACCCTCACTGAATTATCC |
| | nucleic acid | |
| | sequence | |
| 4 | Fasinumab HCDR1, | GFTLTELS |
| | amino acid | |
| | sequence | |
| 5 | Fasinumab HCDR2, | TTTGATCCTGAAGATGGTGAAACA |
| | nucleic acid | |
| | sequence | |
| 6 | fasinumab HCDR2, | FDPEDGET |
| | amino acid | |
| | sequence | |
| 7 | Fasinumab HCDR3, | TCAACGATTTTTGGAGTGGTTACCAACTTTGACAAC |
| | nucleic acid | |
| | sequence | |
| 8 | fasinumab HCDR3, | STIFGVVTNFDN |
| | amino acid | |
| | sequence | |
| 9 | Fasinumab LCVR, | GACATCCAGATGACCCAGTCTCCATCCTCCCTGTCTGCATCTGCAGGAGACAGAGTC |
| | nucleic acid | ACCATCACTTGCCGGGCAAGTCAGGCCATTAGAAATGATTTAGGCTGGTATCAGCAG |
| | sequence | AAACCAGGGAAAGCCCCTAAGCGCCTGATCTATGCTGCATTCAATTTGCAAAGTGGG |
| | | GTCCCATCAAGATTCAGCGGCAGTGGATCTGGGACAGAATTCACTCTCACAATCAGT |
| | | AGCCTGCAGCCTGAAGATCTTGCAAGTTATTACTGTCAACAGTATAATAGATACCCG |
| | | TGGACGTTCGGCCAAGGGACCAAGGTGGAAATCAAACGA |
| 10 | Fasinumab LCVR, | DIQMTQSPSSLSASAGDRVTITCRASQAIRNDLGWYQQKPGKAPKRLIYAAFNLQSG |
| | amino acid | VPSRFSGSGSGTEFTLTISSLQPEDLASYYCQQYNRYPWTFGQGTKVEIKR |
| | sequence | |
| 11 | Fasinumab LCDR1, | CAGGCCATTAGAAATGAT |
| | nucleic acid | |
| | sequence | |
| 12 | Fasinumab LCDR1, | QAIRND |
| | amino acid | |
| | sequence | |
| 13 | Fasinumab LCDR2, | GCTGCATTC |
| | nucleic acid | |
| | sequence | |
| 14 | Fasinumab LCDR2, | AAF |
| | amino acid | |
| | sequence | |
| 15 | Fasinumab LCDR3, | CAACAGTATAATAGATACCCGTGGACG |
| | nucleic acid | |
| | sequence | |
| 16 | Fasinumab LCDR3, | QQYNRYPWT |
| | amino acid | |
| | sequence | |
| 17 | Fasinumab heavy | QVQLVQSGAEVKKPGASVKVSCKVSGFTLTELSIHWVRQAPGKGLEWMGGFDPEDGET |
| | chain, amino | IYAQKFQGRVTMTEDTSTDTAYMELTSLRSEDTAVYYCSTIFGVVTNFDNWGQGTLVT |
| | acid | VSSASTKGPSVFPLAPCSRSTSESTAALGCLVKDYFPEPVTVSWNSGALTSGVHTFPA |
| | sequence | SVLQSGLYSLSSVVTVPSSSLGTKTYTCNVDHKPSNTKVDKRVESKYGPPCPPCPAPE |
| | | FLGGPSVFLFPPKPKDTLMISRTPEVTCVVVDVSQEDPEVQFNWYVDGVEVHNAKTKP |
| | | REEQFNSTYRVVSVLTVLHQDWLNGKEYKCKVSNKGLPSSIEKTISKAKGQPREPQVY |
| | | TLPPSQEEMTKNQVSLTCLVKGFYPSDIAVEWESNGQPENNYKTTPPVLDSDGSFFLY |
| | | SRLTVDKSRWQEGNVFSCSVMHEALHNHYTQKSLSLSLGK |
| 18 | Fasinumab light | DIQMTQSPSSLSASAGDRVTITCRASQAIRNDLGWYQQKPGKAPKRLIYAAFNLQSGVP |
| | chain, amino | SRFSGSGSGTEFTLTISSLQPEDLASYYCQQYNRYPWTFGQGTKVEIKRTVAAPSVFIF |
| | acid | PPSDEQLKSGTASVVCLLNNFYPREAKVQWKVDNALQSGNSQESVTEQDSKQSTYSLSS |
| | sequence | TLTLSKADYEKHKVYACEVTHQGLSSPVTKSFNRGEC |
| 19 | human NGF, | AGCGTCCGGACCCAATAACAGTTTTACCAAGGGAGCAGCTTTCTATCCTGGCCACAC |
| | nucleic acid | TGAGGTGCATAGCGTAATGTCCATGTTGTTCTACACTCTGATCACAGCTTTTCTGATC |
| | sequence | GGCATACAGGCGGAACCACACTCAGAGAGCAATGTCCCTGCAGGACACACCATCCC |
| | | CCAAGCCCACTGGACTAAACTTCAGCATTCCCTTGACACTGCCCTTCGCAGAGCCCGC |
| | | AGCGCCCCGGCAGCGGCGATAGCTGCACGCGTGGCGGGGCAGACCCGCAACATTAC |
| | | TGTGGACCCCAGGCTGTTTAAAAAGCGGCGACTCCGTTCACCCCGTGTGCTGTTTAG |
| | | CACCCAGCCTCCCCGTGAAGCTGCAGACACTCAGGATCTGGACTTCGAGGTCGGTGG |
| | | TGCTGCCCCCTTCAACAGGACTCACAGGAGCAAGCGGTCATCATCCCATCCCATCTTC |
| | | CACAGGGGCGAATTCTCGGTGTGTGACAGTGTCAGCGTGTGGGTTGGGGATAAGAC |
| | | CACCGCCACAGACATCAAGGGCAAGGAGGTGATGGTGTTGGGAGAGGTGAGCATT |
| | | AACAACAGTGTATTCAAACAGTACTTTTTTGAGACCAAGTGCCGGGACCCAAATCCC |
| | | GTTGACAGCGGGTGCCGGGGCATTGACTCAAAGCACTGGAACTCATATTGTACCACG |
| | | ACTCACACCTTTGTCAAGGCGCTGACCATGGATGGCAAGCAGGCTGCCTGGCGGTTT |
| | | ATCCGGATAGATACGGCCTGTATGTGTGTGCTCAGCAGGAAGGCTGTGAGAAGAGC |
| | | CTGACCTGCCGACACGCTCCCTCCCCCTGCCCCTTCTACACTCTCCTGGGCC |
| 20 | human NGF, | SSSHPIFHRGEFSVVSVWVGDKTTATDIKGKEVMVLGEVNINNSVFKQYFFETKCR |
| | amino acid | DPNPVDSGCRGIDSKHWNSYCTTTHTFALTMDGKQAAWRFIRIDTACVCVLSRKAV |
| | sequence | RRA |

1. A method for treating or reducing pain associated with osteoarthritis of the knee and/or hip in a subject, the method comprising:
(a) selecting a subject having osteoarthritis of the knee or hip, wherein the subject to be treated has no more than 2 large joints that exhibit osteoarthritis, wherein the large joints are selected from the group consisting of knee joint, hip joint, and shoulder joint;
(b) administering to the subject one or more doses of an anti-NGF antibody or antigen-binding fragment thereof;
(c) determining whether the subject is a candidate for continued treatment with the anti-NGF antibody or antigen-binding fragment thereof, comprising comparing the level of alkaline phosphatase in the subject at a timepoint after the start of treatment with the anti-NGF antibody to a baseline level of alkaline phosphatase in the subject prior to or at the start of treatment, wherein a subject is identified as a candidate for continued treatment if the subject does not have an increase in the level of alkaline phosphatase that is above a threshold value; and
(d) administering to the subject who is identified as a candidate for continued treatment one or more additional doses of the anti-NGF antibody or antigen-binding fragment thereof.

2. The method of claim 1, wherein the threshold value in step (c) is a 15-point increase in the level of alkaline phosphatase, relative to the baseline level of alkaline phosphatase.

3. The method of claim 1, wherein the threshold value in step (c) is a 10-point increase in the level of alkaline phosphatase, relative to the baseline level of alkaline phosphatase.

4. The method of claim 1, wherein the threshold value in step (c) is a 5-point increase in the level of alkaline phosphatase, relative to the baseline level of alkaline phosphatase.

5. The method of claim 1, wherein the baseline level of alkaline phosphatase is an average of two or more measurements of alkaline phosphatase for the subject, wherein each measurement is taken prior to or at the start of treatment.

6. The method of claim 1, wherein in step (c) the timepoint is about 8 weeks to about 16 weeks from the start of treatment with the anti-NGF antibody.

7. The method of claim 6, wherein in step (c) the timepoint is about 8 weeks from the start of treatment with the anti-NGF antibody.

8. The method of claim 1, wherein step (c) further comprises measuring the level of pain in the subject and identifying the subject as a candidate for continued treatment if the subject exhibits a decrease in pain at a timepoint after the start of treatment relative to a baseline level of pain in the subject prior to or at the start of treatment.

9. The method of claim 8, wherein the level of pain is measured using:
(a) the Western Ontario and McMaster Universities Osteoarthritis Index (WOMAC) pain subscale score;
(b) the Numeric Rating Scale (NRS) for joint pain; and/or
(c) the Patient Global Assessment (PGA) of knee and/or hip pain.

10. The method of claim 9, wherein the subject is identified as a candidate for continued treatment if the subject exhibits a ≥30% improvement in WOMAC pain subscale score relative to the baseline level.

11. The method of claim 1, wherein in step (a) the number of joints that exhibit osteoarthritis is determined by X-ray.

12. The method of claim 1, further comprising:
(e) comparing the level of alkaline phosphatase in the subject at a second timepoint to the baseline level of alkaline phosphatase in the subject, wherein the second timepoint is after the administration of the one or more additional doses of the anti-NGF antibody or antigen-binding fragment thereof according to step (d).

13. The method of claim 12, wherein the second timepoint is at least 8 weeks after the administration of the one or more additional doses of the anti-NGF antibody or antigen-binding fragment thereof.

14. The method of claim 13, wherein the second timepoint is about 8 weeks to about 16 weeks after the administration of the one or more additional doses of the anti-NGF antibody or antigen-binding fragment thereof.

15. The method of claim 1, wherein the anti-NGF antibody or antigen-binding fragment thereof is administered every four weeks (Q4W) or every eight weeks (Q8W).

16. A method for treating or reducing pain associated with osteoarthritis of the knee and/or hip in a subject having osteoarthritis, the method comprising:
(a) administering to the subject an initial dose of an anti-NGF antibody or antigen-binding fragment thereof;
(b) administering to the subject one or more secondary doses of the anti-NGF antibody or antigen-binding fragment thereof, wherein each secondary dose is administered 4 weeks or 8 weeks after the immediately preceding dose;
(c) obtaining a measurement of alkaline phosphatase level in the subject at a timepoint from 8 weeks to 16 weeks after the administration of the initial dose; and
(d) administering to the subject one or more tertiary doses of the anti-NGF antibody or antigen-binding fragment thereof only if the subject's alkaline phosphatase level in step (c) is not increased above a threshold value, relative to a baseline level of alkaline phosphatase in the subject prior to or at the start of treatment; wherein each tertiary dose is administered 4 weeks or 8 weeks after the immediately preceding dose.

17. The method of claim 16, wherein the threshold value in step (d) is a increase in the level of alkaline phosphatase, relative to the baseline level of alkaline phosphatase.

18. The method of claim 16, wherein the threshold value in step (c) is a increase in the level of alkaline phosphatase, relative to the baseline level of alkaline phosphatase.

19. The method of claim 16, wherein the threshold value in step (c) is a 5-point increase in the level of alkaline phosphatase, relative to the baseline level of alkaline phosphatase.

20. The method of claim 16, wherein the baseline level of alkaline phosphatase is an average of two or more measurements of alkaline phosphatase for the subject, wherein each measurement is taken prior to or at the start of treatment.

21. The method of claim 16, wherein in step (c) the timepoint is about 8 weeks to about 16 weeks from the start of treatment with the anti-NGF antibody.

22. The method of claim 1, wherein the alkaline phosphatase is serum alkaline phosphatase.

23. The method of claim 22, wherein the alkaline phosphatase is bone-specific alkaline phosphatase.

24. The method of claim 1, wherein the alkaline phosphatase is measured using an enzymatic assay.

25. A method for treating or reducing pain associated with osteoarthritis of the knee and/or hip in a subject having osteoarthritis, the method comprising:
(a) selecting a subject having osteoarthritis of the knee or hip, wherein the subject to be treated has no more than 2 large joints that exhibit osteoarthritis, wherein the large joints are selected from the group consisting of knee joint, hip joint, and shoulder joint; and
(b) administering to the subject an anti-NGF antibody or antigen-binding fragment thereof, wherein the anti-NGF antibody or antigen-binding fragment thereof is administered every four weeks (Q4W) or every eight weeks (Q8W).

26. The method of claim 25, wherein in step (a) the number of joints that exhibit osteoarthritis is determined by X-ray.

27. The method of claim 1, wherein the subject to be treated does not have a pre-existing subchondral insufficiency fracture (SIF) or osteonecrosis.

28. The method of claim 1, wherein the subject is resistant, non-responsive, or inadequately responsive to treatment with a standard analgesic, or wherein the subject has an intolerance to standard analgesic therapy.

29. The method of claim 28, wherein the standard analgesic therapy is acetaminophen/paracetamol, a nonsteroidal anti-inflammatory drug (NSAID), an opioid, or a combination thereof.

30. The method of claim 1, wherein the subject to be treated has an osteoarthritis polygenic risk score (OA-PRS) that is less than a threshold OA-PRS, wherein the OA-PRS comprises a weighted aggregate of a plurality of genetic variants associated with osteoarthritis.

31. The method of claim 1, wherein the anti-NGF antibody or antigen-binding fragment thereof comprises three heavy chain complementarity determining region (HCDR) sequences (HCDR1, HCDR2, and HCDR3) comprising the amino acid sequences of SEQ ID NOs: 4, 6, and 8, respectively, and three light chain complementarity determining (LCDR) sequences (LCDR1, LCDR2, and LCDR3) comprising the amino acid sequences of SEQ ID NOs: 12, 14, and 16, respectively.

32. The method of claim 31, wherein the anti-NGF antibody or antigen-binding fragment thereof comprises a heavy chain variable region (HCVR) comprising the amino acid sequence of SEQ ID NO:2 and a light chain variable region (LCVR) comprising the amino acid sequence of SEQ ID NO:10.

33. The method claim 32, wherein the anti-NGF antibody or antigen-binding fragment thereof comprises a heavy chain comprising the amino acid sequence of SEQ ID NO:17 and/or a light chain comprising the amino acid sequence of SEQ ID NO:18.

34. The method of claim 33, wherein the anti-NGF antibody is fasinumab.

35. The method of claim 1, wherein the anti-NGF antibody or antigen-binding fragment thereof is tanezumab or fulranumab.

36. The method of claim 1, wherein the anti-NGF antibody or antigen-binding fragment thereof is administered at a dose from 0.5 mg to 10 mg.

37. The method of claim 36, wherein the anti-NGF antibody or antigen-binding fragment thereof is administered at a dose of about 1 mg.


SEQUENCE LISTING (20 sequences)

SEQ ID NO: 1 - DNA, 357 bp, synthetic construct
  misc_feature 1..357: note = synthetic
  source 1..357: mol_type = other DNA
caggtgcagctggtgcagtctggggctgaggtgaagaagcctggggcctcagtgaaggtctcctgcaaggtttccggattcaccctcactgaattatccattcactgggtgcgacaggctcctggaaaagggcttgagtggatgggaggttttgatcctgaagatggtgaaacaatctacgcacagaagttccagggcagagtcaccatgaccgaggacacatctacagacacagcctacatggagctgaccagcctgagatcggaagacacggccgtgtattactgttcaacgatttttggagtggttaccaactttgacaactggggccagggaaccctggtcaccgtctcctca

SEQ ID NO: 2 - protein, 119 aa, synthetic construct [sequence omitted]

SEQ ID NO: 3 - DNA, 24 bp, synthetic construct
  misc_feature 1..24: note = synthetic
  source 1..24: mol_type = other DNA
ggattcaccctcactgaattatcc

SEQ ID NO: 4 - protein, 8 aa, synthetic construct
  REGION 1..8: note = synthetic
GFTLTELS

SEQ ID NO: 5 - DNA, 24 bp, synthetic construct
  misc_feature 1..24: note = synthetic
  source 1..24: mol_type = other DNA
tttgatcctgaagatggtgaaaca

SEQ ID NO: 6 - protein, 8 aa, synthetic construct
  REGION 1..8: note = synthetic
FDPEDGET

SEQ ID NO: 7 - DNA, 36 bp, synthetic construct
  misc_feature 1..36: note = synthetic
  source 1..36: mol_type = other DNA
tcaacgatttttggagtggttaccaactttgacaac

SEQ ID NO: 8 - protein, 12 aa, synthetic construct
  REGION 1..12: note = synthetic
STIFGVVTNFDN

SEQ ID NO: 9 - DNA, 324 bp, synthetic construct
  misc_feature 1..324: note = synthetic
  source 1..324: mol_type = other DNA
gacatccagatgacccagtctccatcctccctgtctgcatctgcaggagacagagtcaccatcacttgccgggcaagtcaggccattagaaatgatttaggctggtatcagcagaaaccagggaaagcccctaagcgcctgatctatgctgcattcaatttgcaaagtggggtcccatcaagattcagcggcagtggatctgggacagaattcactctcacaatcagtagcctgcagcctgaagatcttgcaagttattactgtcaacagtataatagatacccgtggacgttcggccaagggaccaaggtggaaatcaaacga

SEQ ID NO: 10 - protein, 108 aa, synthetic construct
  REGION 1..108: note = synthetic
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKVEIKR

SEQ ID NO: 11 - DNA, 18 bp, synthetic construct
  misc_feature 1..18: note = synthetic
  source 1..18: mol_type = other DNA
caggccattagaaatgat

SEQ ID NO: 12 - protein, 6 aa, synthetic construct
  REGION 1..6: note = synthetic
QAIRND

SEQ ID NO: 13-14 - skipped (2 entries)

SEQ ID NO: 15 - DNA, 27 bp, synthetic construct
  source 1..27: mol_type = other DNA
  misc_feature 1..27
caacagtataatagatacccgtggacg

SEQ ID NO: 16 - protein, 9 aa, synthetic construct
  REGION 1..9: note = synthetic
QQYNRYPWT

SEQ ID NO: 17 - protein, 446 aa, synthetic construct
QVQLVQSGAEVKKPGASVKVSCKVSGFTLTELSIHWVRQAPGKGLEWMGGFDPEDGETIYAQKFQGRVTMTEDTSTDTAYMELTSLRSEDTAVYYCSTIFGVVTNFDNWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - protein, 214 aa, synthetic construct
DIQMTQSPSSLSASAGDRVTITCRASQAIRNDLGWYQQKPGKAPKRLIYAAFNLQSGVPSRFSGSGSGTEFTLTISSLQPEDLASYYCQQYNRYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - DNA, 847 bp, Homo sapiens
  source 1..847: mol_type = genomic DNA
agcgtccggacccaataacagttttaccaagggagcagctttctatcctggccacactgaggtgcatagcgtaatgtccatgttgttctacactctgatcacagcttttctgatcggcatacaggcggaaccacactcagagagcaatgtccctgcaggacacaccatcccccaagcccactggactaaacttcagcattcccttgacactgcccttcgcagagcccgcagcgccccggcagcggcgatagctgcacgcgtggcggggcagacccgcaacattactgtggaccccaggctgtttaaaaagcggcgactccgttcaccccgtgtgctgtttagcacccagcctccccgtgaagctgcagacactcaggatctggacttcgaggtcggtggtgctgcccccttcaacaggactcacaggagcaagcggtcatcatcccatcccatcttccacaggggcgaattctcggtgtgtgacagtgtcagcgtgtgggttggggataagaccaccgccacagacatcaagggcaaggaggtgatggtgttgggagaggtgagcattaacaacagtgtattcaaacagtacttttttgagaccaagtgccgggacccaaatcccgttgacagcgggtgccggggcattgactcaaagcactggaactcatattgtaccacgactcacacctttgtcaaggcgctgaccatggatggcaagcaggctgcctggcggtttatccggatagatacggcctgtatgtgtgtgctcagcaggaaggctgtgagaagagcctgacctgccgacacgctccctccccctgccccttctacactctcctgggcc

SEQ ID NO: 20 - protein, 115 aa, Homo sapiens
SSSHPIFHRGEFSVVSVWVGDKTTATDIKGKEVMVLGEVNINNSVFKQYFFETKCRDPNPVDSGCRGIDSKHWNSYCTTTHTFALTMDGKQAAWRFIRIDTACVCVLSRKAVRRA